(19) United States
(12) Patent Application Publication
(10) Pub. No.: US 2009/0203636 A1
(43) Pub. Date: Aug. 13, 2009

(54) Prevention and Treatment of Cancer and Other Diseases

(76) Inventor: Igor E. Bondarev, St. Petersburg (RU)

Correspondence Address: Nanda P B A Kumar;Reed Smith, 1650 Market Street, Philadelphia, PA, 19103, US

(21) Appl. No.: 12/225,199

(22) PCT Filed: Mar. 14, 2007
(86) PCT No.: PCT/US2007/006538

§ 371 Date: Jan. 29, 2009

Related U.S. Application Data
(60) Provisional application No. 60/782,559, filed on Mar. 14, 2006.
(60) Provisional application No. 60/801,698, filed on May 20, 2006.
(60) Provisional application No. 60/860,518, filed on Nov. 21, 2006.

(51) Int. Cl. A61K 31/708; A61K 31/7068; A61P 35/00; A61K 31/513; A61K 31/52; C07D 473/34; C07D 239/52

(52) U.S. Cl. 514/45; 514/49; 514/274; 514/263.4; 544/277; 544/314

(57) ABSTRACT

Nucleoside chemical compounds, which interact with specific structures of deoxyribonucleic acid (DNA) or ribonucleic acid (RNA) are disclosed. The compounds interfere with the activities of telomerase and reverse transcriptase, and are useful as antivirals, antibacterials and anticancer agents. Methods of treating or preventing cancers in patients involving administration of a therapeutically effective amount of a composition having an inhibitor or antagonist of the reverse transcriptases (RTs) expressed in cells of the patients are also disclosed. Method of using nucleoside analogs and other inhibitors of RTs in conjunction with DNA damaging agents such as genotoxic agents or radiation or photodynamic therapy or combinations these for the treatment of various cancers are also disclosed.

This application claims the benefit of U.S. Provisional Application No. 60/782,559 filed Mar. 14, 2006, US. Provisional Application No. 60/801,693 filed May 18, 2006 and U.S. Provisional Application No. 60/860,518 filed Nov. 21, 2006, and is a continuation-in-part of PCT/US2006/019488, filed May 18, 2006 and the text of applications 60/782,559, 60/801,693, 60/860,518 and PCT/US2006/019488 is incorporated by reference in its entirety herewith.

### FIELD OF THE INVENTION

The present invention is directed to the field of cancer therapy and in particular methods of using a combination of inhibitors of reverse transcriptases (RTs) for inhibition of growth of cancer cells and treatment and prevention of cancers. The present invention also involves methods of using nucleoside analogs and other inhibitors of RTs in conjunction with DNA damaging agents such as genotoxic agents or radiation or photodynamic therapy or combinations of these for the treatment of various cancers. The present invention also relates to novel nucleoside chemical compounds which interact with specific structures of deoxyribonucleic acid (DNA) or ribonucleic acid (RNA). More specifically, the compounds of the present invention interfere with the activities of telomerase and reverse transcriptase and are useful as antivirals, antiparasiticals, antibacterials and anticancer agents.

### BACKGROUND OF THE INVENTION

Cell division (proliferation) is a physiological process that occurs in almost all tissues and under many circumstances. Progression through the cell cycle is controlled by the combined effects of kinases, phosphatases and inhibitory proteins mediated by protein partnering and positive- and negative-acting phosphorylation. Cell cycle progression is characterized by checkpoints where the cell determines whether previous steps have been successfully completed before moving forward. Most cells have a fixed number of divisions (approximately 50) before they die. The PCT application publication WO 2005/069880 describes how cells enter mortality stage (M1 and M2) and circumstances under which some cells escape the mortality stage, and maintain the ability to divide rapidly in an uncontrolled manner through telomere maintenance.

The uncontrolled and often rapid proliferation of cells can lead to the formation of tumors (benign or malignant). Benign tumors do not spread to other parts of the body or invade other tissues, and they are rarely a threat to life unless they compress vital structures or are physiologically active (for instance, producing a hormone such as estrogen). Malignant tumors can invade other organs, spread to distant locations (metastasize) and become life threatening. Cells capable of forming malignant tumors exhibit an uncontrolled ability to divide (or, they are immortal), and they often divide at an increased rate compared to the cells of healthy tissue or even benign tumors. Therefore, cancer therapies often focus on eliminating malignant cells.

Traditionally, the efficacy of many cancer therapies was believed to arise from the cytotoxicity derived from chemotherapy-induced and/or radiation-induced DNA damage. Such DNA damage was considered to trigger an apoptotic response. For example, capecitabine (also called Xeloda®) and anthracycline daunorubicin (also called DNR) therapies were believed to induce cytotoxicity as a result of drug-induced damage to DNA.

In recent years, more targeted approaches that interfere with telomere maintenance and causing cell cycle arrest and apoptosis in cancer cells have been established. Elongation of shortened telomeres by telomerase is a well known mechanism of telomere maintenance in the human cancer cells. Telomersse maintains telomeric DNA and plays a critical role in tumor cell immortality. Human telomerase is repressed or transiently active in normal somatic cells and telomeres gradually shorten over decades. It has been reported that in most cancers, telomeres (though short) are maintained by telomerase. A correlation between telomerase activation and tumor progression has been demonstrated. This has led skilled artisan to believe that the inhibition of telomerase as a promising approach for the treatment of cancer. However, up to 30% of human tumors of different types do not express telomerase. The presence of non-telomerase mediated telomere maintenance or alternative lengthening of telomeres was reported in up to 30% of human tumors of different types, tumor-derived cell lines and human cell lines immortalized in vitro. Bryan et al., Nat. Med. 3:1271-1274, 1997; Reddel et al., Radiat. Res. 155:194-200, 2001; Bryan et al., Eur. J. Cancer 33:767-773, 1997; Bryan et al., EMBO J. 14: 4240-4248,1995) and up to 50% in some subsets of tumors and immortalized cell lines (Gupta et al., J. Natl. Cancer Inst. 88:1152-1157 (1996).

The PCT application publication WO 2005/069880 reports that certain nucleoside analogs can induce apoptosis in telomerase negative cancer cells. For example, when U-2 OS and Saos-2 osteosarcoma cells when treated with therapeutic concentrations of AZT or ganciclovir (GCV), these nucleoside analogs induced telomere shortening, G2 arrest and massive apoptosis in these cancer cells after 14 days of treatment. Likewise, the U.S. Pat. No. 6,723,712 reports certain nucleoside analogs for use in treatment of cancer. Specifically, it reports that the combination of an anti-viral nucleoside phosphate analogue, cidofovir, and irradiation as an approach for the treatment of human cancers. In particular, it reports that when Ramos (lymphoma) HTB31 and SCC97 (carcinoma) cells were treated with irradiation alone and cidofovir, both irradiation alone and cidofovir alone induced a weak growth delay, whereas the concomitant association of both agents dramatically reduced the growth delay for the tumor cells studied. In addition, Sciamanna et al., 2005, Oncogene 24:3923, report that the endogenous reverse transcriptase (RT) or non-telomerase RT can be an epigenetic regulator of cell proliferation and inhibition of RT activity in vivo antagonize tumor growth in animal experiments.

These reports suggest that therapies for inhibiting cancer cell growth are steadily evolving. However, it remains a fact that even the best current therapies are not always sufficiently effective and/or frequently become ineffective after treatment, and are frequently accompanied by significant side effects, so that improved anticancer therapies are constantly being sought. Synthetic nucleoside analogs are known to be therapeutically useful, among others, as antivirals, antibiotics, and anticancer agents. Many of these nucleoside analogs are on the market. However, the increasing resistance of pathogens and the often severe side effects of nucleosides in chemotherapy despite extensive medicinal chemistry research emphasize the need for nucleoside analogs in high number and diversity. In particular, a need continues to exist for methods and agents for achieving sufficient control over the abnormal cell proliferation including abnormal growth potential of cancer cells and cancer cure.

### SUMMARY OF THE INVENTION

The present invention fulfills this need by providing methods and related compounds in a certain combination for treating conditions characterized by abnormal cell proliferation, including, but not limited to, cancer and metastasis.

The invention also discloses novel acyclic nucleoside analogs useful as chain terminators in enzymatic nucleic acid synthesis/elongation reactions.

The invention is based, in part, on the discovery that the simultaneous inhibition of telomere maintenance mechanisms (TMMs) leads to progressive telomere shortening and G2/M phase arrest of cell cycle in cancer cells thereby limiting the proliferation potential of cells. It has also been shown that in the absence of such simultaneous inhibition, the cells continue to proliferate abnormally by switching from one telomere maintenance mechanism to another. For simultaneous inhibition of telomere maintenance mechanisms, a combination of compounds (inhibitors of several reverse trascriptases) is used. Further, it has also been discovered by the present inventor that the simultaneous inhibition of TMMs makes cancer cells more sensitive to any kind of DNA damaging therapy (e.g., genotoxic chemotherapy, radiotherapy, photodynamic therapy). More specifically, it has been discovered that the inhibition of telomere maintenance, leading to telomere shortening and G₂/M phase arrest in cancer cells, could not only limit the abnormal proliferation of cells but also increase the efficacy of DNA damaging agents because the cells are most sensitive to such agents perhaps due to G₂/M phase arrest.

In one aspect, the invention provides a method for treating a subject having a condition characterized by abnormal mammalian cell proliferation. The method comprises administering to a subject in need of such treatment, telomere maintenance affecting (or telomere shortening) combination of compounds in an amount effective to inhibit the proliferation, wherein the combination is a double cocktail combination or a triple cocktail combination.

According to one embodiment, the subjects are treated with a given cocktail of compounds in a manner and in an amount so as to inhibit proliferation of a primary tumor, or to inhibit metastatic spread or growth while minimizing the potential for systemic toxicity particularly from the use of other DNA damaging agents. In certain embodiments, the abnormal mammalian cell proliferation is manifested as a tumor. Some conditions intended to be treated by the method of the invention include benign (i.e., non-cancerous), pre-malignant and malignant (i.e., cancerous) tumors. In some embodiments, the condition characterized by abnormal mammalian cell proliferation is further characterized by the presence of cells with long telomeres as compared to their normal counterparts over successive cell divisions.

In other embodiments, the abnormal mammalian cell proliferation may be a condition that is diagnosed as a carcinoma, a sarcoma, and a melanoma. In yet other embodiments, the condition is any of colorectal cancer, pancreatic cancer, lung cancer, breast cancer, ovarian cancer, prostate cancer, kidney cancer, melanoma and fibrosarcoma. In still other embodiments, the condition may be one related to bone and connective tissue sarcomas, examples of which include, but are not limited to, osteosarcoma and fibrosarcoma.

In some other embodiments, the abnormal mammalian cell proliferation is in epithelial cells. Some conditions characterized by abnormal mammalian epithelial cell proliferation include adenomas of epithelial tissues such as the breast, colon and prostate, as well as malignant tumors. According to other embodiments of the invention, a method is provided for treating a subject having a metastasis of epithelial origin.

As described above, the subjects to be treated are subjects having a condition characterized by abnormal mammalian cell proliferation or cancer. In certain embodiments, however, the subjects are free of abnormal mammalian cell proliferation or cancer but are likely to develop such conditions (based on certain biomarkers or genetic defects) thereby calling for treatment with a combination of compounds for telomere shortening and G₂/M phase arrest.

In another aspect of the invention, a method is provided in which a combination of compounds capable of affecting telomere maintenance is administered in combination with one or more DNA-damaging agents such as a genotoxic chemotherapeutic agent. In another embodiment, a combination of compounds with or without DNA-damaging agent(s) is administered in combination with surgery to remove an abnormal proliferative cell mass. In a related embodiment, a combination of compounds with or without DNA-damaging agent(s) is administered to a patient who has had surgery to remove an abnormal proliferative cell mass.

In some embodiments, the abnormal mammalian cell proliferation is manifested as a tumor. In another embodiment, the abnormal mammalian cell proliferation is selected from the group consisting of a carcinoma, a sarcoma, and a melanoma. In still another embodiment, the condition characterized by abnormal mammalian cell proliferation is a metastasis. In other embodiments, the condition is selected from the group consisting of breast cancer, colorectal cancer, ovarian cancer, prostate cancer, pancreatic cancer, kidney cancer, lung cancer, melanoma and fibrosarcoma. In another embodiment, the abnormal mammalian cell proliferation is in epithelial cells, meaning that epithelial cells are abnormally proliferating.

The combination of compounds or compositions thereof may all be administered in a systemic manner, via administration routes such as, but not limited to, oral, intravenous, intramuscular and intraperitoneal administration. In some instances, however, a combination containing three different compounds, two may be administered systemically while the third is administered by other routes. Systemic administration routes may be preferred, for example, if the subject has metastatic lesions.

Some or all of the compounds of a given combination may also be administered locally. In some embodiments, the compounds or compositions containing the compounds are targeted to a tumor. This can be achieved by the particular mode of administration. For example, easily accessible tumors such as breast or prostate tumors may be targeted by direct needle injection to the site of the lesion. Lung tumors may be targeted by the use of inhalation as a route of administration.

Although not necessary, in some embodiments, one or more compounds of a given combination may be targeted to a cell mass (e.g., a tumor) through the use of a targeting compound specific for a particular tissue or tumor type. In some embodiments, the compounds may be targeted to primary or in some instances, secondary (i.e., metastatic) lesions through the use of targeting compounds which preferentially recognize a cell surface marker. In other embodiments, one or more compounds of a given combination may also be administered in a sustained release formulation.

Accordingly, in one aspect of the invention, thymine and adenine derivatives of the formulas (I), (II), (III), (IV), (V) and (VI) are disclosed. Physiologically acceptable salts, optical isomers and pro-drugs of formulas (I), (II), (D), (IV), (V) and (VI) are disclosed. In one embodiment, the thymine derivative is 1-(2-hydroxyethoxymethyl) and the adenine derivative is 9-(2-hydroxyethoxymethyl) adenine.

Pharmaceutical preparations having, as an active ingredient, a compound of the formula (I), (II), (III), (IV), (V) or (VI) in conjunction with a pharmaceutically acceptable carrier are also disclosed.

In another aspect of the invention, a method for the treatment of cancer in an animal or human patient is disclosed. It involves administering a therapeutically effective amount of a composition having as an active ingredient a compound of the formula (I), (II), (III), (IV), (V) or (VI) or a physiologically acceptable salt or an optical isomer thereof in conjunction with azido-2′,3′-dideoxythymidine (AZT) and 2′,3′-deoxyinosine (didanosine or ddI) in a pharmaceutically acceptable carrier. The composition can further include a therapeutically effective amount of a composition having an anticancer agent such as, for example, cyclophosphamide, capecitabine, taxol, cisplatin, carboplatin, camptothecins and/or doxorubicin.

In addition to the therapeutic aspect based on the use of nucleoside analogs that are acyclic, anti-telomerase, anti-L1RT and antineoplastic, the present invention also provides diagnostic methods and kits for detecting pathologically proliferating cells expressing telomerase or L1RT. These and other aspects of the invention will be described in greater detail below. Throughout this disclosure, all technical and scientific terms have the same meaning as commonly understood by one of ordinary skill in the art to which this invention pertains unless defined otherwise.

### DETAILED DESCRIPTION OF THE INVENTION

This invention concerns methods and compositions for shortening telomeres in proliferating cells. This invention also concerns methods and compositions for inhibition of growth of proliferating cells. Shortening of telomeres or inhibition of cell growth is accomplished by interfering with telomere maintenance mechanisms, more particularly by interfering with the activity of reverse transcriptases (RTs) in such cells. A combination of compounds is used for these purposes. The compounds shorten telomeres or affect telomere maintenance and as a consequence affect the growth properties and/or cause the death of pathologically proliferating cells (e.g., cancer cells).

Telomeres play a role in allowing the end of the linear chromosomal DNA to be replicated completely without the loss of terminal bases at the 5′-end of each strand. Immortal cells and rapidly proliferating cells use RTs to add telomeric DNA repeats TTAGGG to chromosomal ends. Inhibition of the RTs can result in the proliferating cells not being able to add telomeres and so they should eventually stop dividing further. The combination of compounds used in the present invention should affect telomere maintenance or induce telomere shortening, G2/M arrest and/or massive apoptosis in cancer cells. As will be evident to those of ordinary skill in the art, this method for affecting telomere maintenance and inhibiting the ability of a cell to proliferate is useful for the treatment of a condition associated with cell proliferation such as in cancer or treatment of germ line cells, which may be useful for contraceptive purpose.

With regard to cancer cells, as described above, telomerase activity has already been known to be involved in telomere maintenance and cell immortality, and cancer cells can be telomerase positive or telomerase negative. It is also known in the art that the telomerase negative cancer cells maintain their telomeres and immortality by alternative mechanisms of telomere lengthening (ALT). Recently, it has been reported that L1 (LINE-1) retrotransposon reverse transcriptase (L1RT) is associated with the lengthening and therefore maintenance of telomeres in telomerase negative cancer cells (see WO 2005/069880). Thus, L1RT is one of the alternative mechanisms of telomere lengthening in telomerase negative cancer cells. These reports suggest that the immortalization of cells involve the activation of telomerase (e.g., hTERT) in telomerase positive cells and L1RT in telomerase negative cells. The targeted therapies focusing on telomerase or L1RT may dramatically reduce the cancer cell growth or tumor growth and thus may be efficacious to some extent. To date, however, no drugs specifically identified as telomerase inhibitors or L1RT inhibitors have been tested as anticancer agents in human patients.

One of the major challenges in cancer treatment today is the effective treatment of a given cancer. A great deal of effort is being directed at finding more and more efficacious treatments for cancer. In that spirit, the present inventor has now discovered that a targeted therapy against telomerase or L1RT may not be sufficient to effectively treat a given tumor because of a cancer cell's ability to switch telomere maintenance mechanisms or to activate a given telomere maintenance mechanism. The switch or activation may be spontaneous, induced by the underlying cancer therapy targeted to a specific telomere maintenance mechanism and/or due to extra genomic instability. For example, a cancer therapy using inhibitors of L1RT may inhibit the activity of that enzyme and this inhibition may initially arrest the growth of cancer cells. However, as can be noted from the present disclosure, cancer cells can rely on or activate other telomere maintenance mechanisms and begin to proliferate in an uncontrolled manner. This is somewhat analogous to drug resistance encountered in traditional cancer therapies.

In the present invention, inhibition of cancer cell growth is achieved by using a combination of compounds capable of affecting the growth properties and/or causing the death of such cells. Although not wishing to be bound by theory, it is believed that simultaneous inhibition of more than one telomere maintenance mechanisms (TMM) activated by cancer cells from time to time can be effective to treat any cancer. The compounds specifically interfere with the activities or expression of several reverse transcriptase (RT) molecules seen in cancer cells and are thereby useful in preventing or treating many types of malignancies. These RTs include telomerase and L1 (LINE-1) retrotransposon encoded reverse transcriptase (L1RT). In addition, it is believed that a cancer cell may activate a non-L1RT reverse transcriptase for lengthening of telomeres (a type of ALT). The non-L1RTs include the long terminal repeat (LTR) retrotransposon encoded RT (or LTR RT) and RT of retroviral origin, which may be endogenous or exogenous to the cancer cells.

The combination of compounds used in the present invention, which by interfering with one or more RTs, affects telomere maintenance or induces telomere shortening, G2/M arrest (also referred to herein as G2 arrest) and/or massive apoptosis in cancer cells. In particular, the compounds of the present invention can provide a highly general method of preventing or treating malignancies, as demonstrated by their ability to inhibit both telomerase positive and telomerase negative human tumor cell lines and tumors that express several types of RTs. More importantly, the inhibitors described in the present invention induce telomere reduction during cell division in tumor cell lines but not in normal cells. The inhibitors also are expected to demonstrate no significant cytotoxic effects in normal cells at the RT inhibitory concentrations of their proposed use. As a result, the inhibitors can be effective in providing treatments that selectively target malignant cells, thus avoiding many of the undesirable adverse effects generally associated with cytotoxic chemotherapeutic agents.

Thus, a cancer therapy using the combination of compounds of the present invention can be said to be a combination-based molecular targeted cancer therapy. The therapy using the combination to affect telomere maintenance or induce telomere shortening is referred to herein as telomere shortening therapy or background therapy. This molecular targeted cancer therapy can be combined with one or more of known other anticancer therapies including cytotoxic chemotherapy, biologic therapy, photodynamic therapy, and radiotherapy and used for the effective treatment of cancer.

Telomere maintenance affecting (or telomere shortening) combination of compounds means a combination of inhibitor(s) of TERT (also referred to as telomerase) and inhibitor(s) of L1RT (the combination referred to as double cocktail) or a combination of inhibitor(s) of telomerase RT, inhibitor(s) of L1RT and inhibitor(s) of non-L1RT (the combination referred to as triple cocktail). The therapy by administration of double cocktail or triple cocktail is referred to herein as background therapy.

The inhibitors or antagonists used as part of the combination of compounds in the present invention are those inhibitors or antagonists that can (1) interact or bind specifically with a given RT (at mRNA or protein or template RNA of the enzyme) to inhibit the RT's expression or activity and/or (2) get incorporated into a telomeric DNA repeat, thereby affecting telomere maintenance or telomere length in cells. For example, nucleoside analog(s) corresponding to one or more nucleotides seen in the telomeric repeat sequence, TTAGGG, can get incorporated into the elongating telomeres and affect telomere maintenance or erode the telomere length as cells go through several rounds of cell proliferation process.

Inhibitor can be any one of small molecules, peptides, dominant negative mutant proteins, antibodies, or antibody fragments, and nucleic acid constructs, antisense constructs, dsRNAs corresponding to a defined target region in the selected RT, oligonucleotides known to one skilled in the art. Inhibitor used should bring about the inhibition of RT or a given RT. As used herein, the term “inhibition of RT” refers to a directly measurable inhibition of reverse transcriptase enzymes telomerase, L1RT and/or non-L1RT as demonstrated, for example, by using a non-radioactive assay system described by Spedding G. 1996, J Mol Recognit., 9(5-63:499-502, or based on the reduction of the average telomere length in all of the cells as demonstrated by using the TRAP assay described by Wege et al., 2003, SYBR Green real-time telomeric repeat amplification protocol for the rapid quantification of telomerase activity, Nucleic Acids Res. 31(2):E3-3, or erosion of individual telomeres (see Lansdorp P M, Heterogeneity in telomere length of human chromosomes, Hum Mol Genet., 1996, 5(5):685-91) or in individual cells using the FISH assay described in the published literature (see Hultdin M et al., 1998, Telomere analysis by fluorescence in situ hybridization and flow cytometry, Nucleic Acids Res., 26(16):3651-3656).

Likewise, one skilled in the art would know how to determine whether a particular cocktail has induced telomere shortening in cells. For example, one may perform a terminal restriction fragment (TRF) analysis in which DNA from tumor cells is analyzed by digestion with restriction enzymes specific for certain sequences. An example of such analysis is described in Vaziri H, 1993, Loss of telomeric DNA during aging of normal and trisomy 21 human lymphocytes, Am J Hum Genet., 52(4):661-7. For example, following digestion of the DNA, gel electrophoresis is performed to separate the restriction fragments according to size. The separated fragments then are probed with nucleic acid probes specific for telomeric sequences to determine the lengths of the terminal fragments containing the telomere DNA of the cells in the sample. By measuring the length of telomeric DNA, one can estimate whether or not a given cocktail is inducing telomere shortening and how long the cocktail should be administered. In addition, during treatment, one can test cells to determine whether a decrease in telomere length over progressive cell divisions is occurring to demonstrate treatment efficacy.

Recently, the use of a novel nanosensor developed for rapid screens of reverse transcriptase (telomerase) activity in biological samples has been reported (Grimm et al., 2004, Cancer Research 64:639-643). The technique utilizes magnetic nanoparticles that, on annealing with telomerase synthesized TTAGGG repeats, switch their magnet state, a phenomenon readily detectable by magnetic readers. High-throughput adaptation of the technique by magnetic resonance imaging reportedly allows processing of hundreds of samples within tens of minutes at ultrahigh sensitivities and quantification of the reverse transcriptase activity. Together, these studies establish that there are assays and tools for rapidly sensing reverse transcriptase activity in biological samples (including tumor cells and tissues) and quantify therapeutic inhibition.

Essentially, in the present invention, the inhibitors are used for inhibiting the growth of cells. For example, when a patient is administered with a given combination of inhibitors, these inhibit the growth of cancer cells by affecting telomere maintenance or by causing progressive telomere shortening, cell cycle arrest in the cells and/or massive apoptosis of the cells. It is believed that a triple cocktail combination would be more efficacious in inhibiting the growth of cancer cells than a double cocktail combination because, as demonstrated herein, the cells that continue to proliferate in the presence of the double cocktail combination can be inhibited by the addition of the inhibitor that make up the triple cocktail combination.

Preferred inhibitors of telomerase are nucleoside analogs. Indeed, nucleoside analogs were among the first compounds shown to be effective against viral infections. Acyclovir is used extensively in the treatment of herpetic infections. The first four anti-HIV drugs to be approved, AZT, ddI, ddC and D4T, were also nucleoside analogs. These nucleoside analogs are progressively phosphorylated to a 5′-triphosphate, which then act as chain terminators in a reverse transcriptase am) reaction.

It has been discovered in the present invention that certain novel thymine and adenine derivatives can interact with specific structures of deoxyribonucleic acid (DNA) or ribonucleic acid (RNA). For example, these derivatives can get incorporated into telomeric repeats in proliferating cells and thereby interfere with telomere maintenance. Telomerase is a highly attractive target for treating cancer cells. Thus, one advantage of the compounds of the present invention, from the therapeutic point of view, is in blocking telomerase activity in pathologically proliferating cells.

Accordingly, in one aspect, the present invention provides compositions and methods involving the use of acyclic nucleoside analogs capable of interfering with telomere elongation in telomerase positive cells. The acyclic nucleoside analogs contemplated in some embodiments of the present invention are those having are those having a purine (or a pyrimidine) skeleton with a tail portion (e.g., 9-(1,3-dihydroxy-2-propoxymethyl group) but lacking the hydroxyl cyclic ring (pentose). In one embodiment, acyclic nucleoside analogs contemplated in the present invention are those having an adenine or a thymine skeleton with a tail portion (e.g., 9-(1,3-dihydroxy-2-propoxymethyl group) but lacking the hydroxyl cyclic ring (pentose). In some embodiments, the purine-based nucleoside analogs of the present invention lack NH₂ group at the second position of the guanine skeleton. A number of acyclic nucleoside analogs are already known in the art. These are, for example, acyclovir, ganciclovir, penciclovir and the corresponding pro drugs, i.e., valacyclovir, valganciclovir and famciclovir, respectively. Acyclovir¹² acts by mimicking a cellular DNA constituent, guanine. That is the “G” in the AT-CG of DNA. Acyclovir (9-[2(hydromethoxy)-methyl]guanine), although structurally similar to “G,” is missing its tail—a hydroxyl “cyclic” ring (pentose) and thus it is “acyclic.” Ganciclovir and penciclovir are also “acyclic” because they too lack the hydroxyl cyclic ring.

In an embodiment of the invention, the tail portion of the acyclic nucleoside analogs of the present invention has at least one hydroxyl group mimicking the 3′- and 5′-hydroxyl groups of the 2′-deoxyribose moiety of nucleosides. The acyclic nucleoside analogs of the present invention have been found to exhibit antitelomerase and antineoplastic properties at clinically acceptable doses and exhibiting only clinically acceptable degree of toxicity.

The compounds of the present invention which meet the intended objective, that is to say, which interact with DNA by incorporating into elongating telomeres in cancer cells and thereby exhibit a telomerase-inhibiting activity, are acyclic nucleoside analogs that are telomerase inhibitors and having specificity to the telomerase as mentioned above. Preferred acyclic nucleoside analogs of the present invention correspond to the following formulas, and their pharmaceutically acceptable salts or esters thereof:

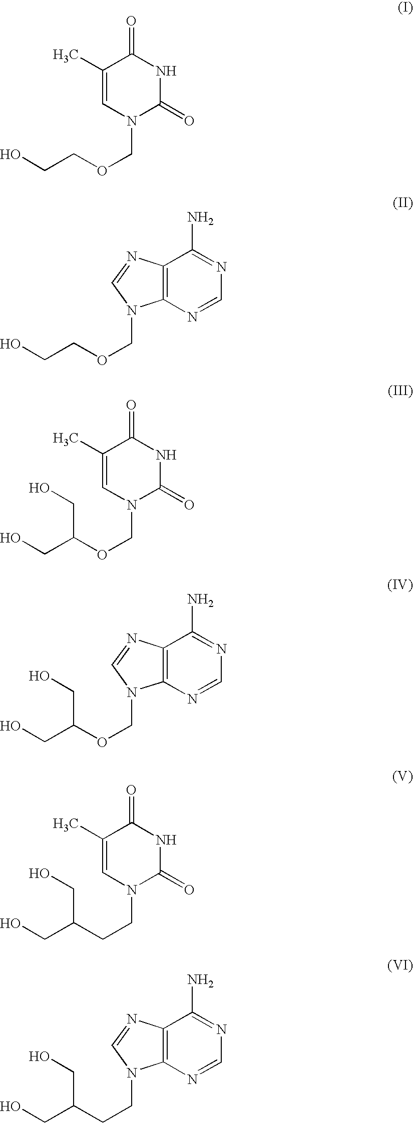

For example, the acyclic nucleoside analogs of the formula (I) or (II), having a tall portion (i.e., 2-hydroxyethoxymethyl group) substituted at the 1-position of thymine or at the 9-position of adenine has a mechanism of action that is quite specific on elongating telomeres. These are specific inhibitors in that these compounds inhibit telomerase mediated telomere elongation but not L1 (LINE-1) retrotransposon reverse transcriptase (L1RT) mediated telomere elongation at least at certain clinically acceptable concentrations. Essentially, the compounds of the present invention are chain terminators. The term “chain terminator” refers to a nucleotide analog that serves as a substrate for a nucleic acid polymerase enzyme, but once incorporated onto the end of a growing polynucleotide chain, the analog cannot itself serve as a substrate for the attachment of subsequent nucleotide residues.

Thus, because of their ability to get incorporated into specific structures of DNA and RNA of cells and viruses and thereby interfere with cellular enzymes (e.g., telomerase) and viral enzymes, the compounds (chain terminators) of the present invention can be therapeutically useful as anticancer agents, antivirals, antibiotics, antipsychotics, analgesic, anti-inflammatory agents, antihypertensives.

The compounds of the present invention can exist in optically active forms, i.e., they have the ability to rotate the plane of plane-polarized light. These include d and 1 or (+) and (−) forms (including stereoisomers, enantiomers, or an enantiomeric mixture). With reference to the instances where (R) or (S) is used, it is to designate the absolute configuration of a substituent in context to the whole compound and not in context to the substituent alone.

The present invention also includes prodrugs. A prodrug, according to the present invention, is one where a parent drug (e.g., SN1) that is an active drug is chemically transformed into a per se inactive derivative, which by virtue of chemical or enzymatic attack is converted to the parent drug within a physiological environment (within the body) before or after reaching the site of action. Stated otherwise, the prodrugs are derivatives of the compounds of the invention which have chemically or metabolically cleavable groups and become, under physiological conditions, the compounds of the invention which are pharmaceutically active in vivo.

Thus, the preparation of a prodrug involves a process of converting an active drug into inactive form. Such processes are well known to one skilled in the art. Prodrugs according to the present invention are those that are carrier-linked-prodrugs and not bioprecursors. The carrier-linked prodrug can result from a temporary linkage of the active molecule with a transport moiety. Such prodrugs are less active or inactive compared to the parent active drug. The transport moiety, which is not limited to any particular any particular chemical or group, will be chosen for its non-toxicity and its ability to ensure the release of the active principle with efficient kinetics. Prodrugs can be prepared, for example, by formation of ester, hemiesters, nitrate esters, amides, carbonate esters, carbamates, lines of the active drug or by functionalizing the drug with azo, glycoside, peptide, and ether functional groups or use of polymers etc., as known to one skilled in the art.

Prodrugs are prepared to alter the drug pharmacokinetics, improve the drug bioavailability by increasing absorption and distribution and decrease toxicity and increase duration of the pharmacological effect of the drug. In designing the prodrugs, one can consider factors such as the linkage between the carrier and the drug is usually a covalent bond, the prodrug is inactive or less active than the active parent, the prodrug is a reversible or bioreversible derivative of the drug, and the carrier moiety is non-toxic and inactive when released.

In one embodiment, prodrugs are prepared by formation of esters of the active drug (e.g., valine esters) or compounds of the formulas I to VI. The carrier moiety (e.g., valine) can be added to the tail portion of the compound of interest. These valine ester compounds, when administered to cells in vitro or in vivo, get converted to the active compound, which is any of the formulas (I) and (VI).

In the present invention it has been shown (cf. working examples below) that the acyclic nucleoside analogs can target telomerase and affect telomere lengthening (or damage telomeres) in cells of a mammal. To target L1RT or other non-telomerase enzymes and affect telomere lengthening (or damage telomeres) in cells of a mammal, the acyclic nucleoside analogs including any of acyclovir, ganciclovir, penciclovir and/or compounds of the formulas I to VI or the corresponding pro-drugs (e.g., valine esters, i.e., valacyclovir, valganciclovir and famciclovir esters of the active drug) and/or other nucleoside analogs such as AZT and ddI can be used.

The nucleoside analogs such as acyclovir, ganciclovir, penciclovir and the corresponding pro-drugs, i.e., valacyclovir, valganciclovir and famciclovir, are all approved for clinical use as antiviral drugs. For example, acyclovir, ganciclovir, penciclovir and the corresponding pro-drugs are well known medicines for the treatment of or relief from Herpes virus or/and CMV infections, their use in therapy of neoplastic diseases is unknown Penciclovir is used on the lips and faces of humans to treat cold sores caused by herpes simplex virus. It is also known in the art that the target enzyme for these nucleoside analogs is the DNA polymerase. Their chemical structures and dosage regimens for combating viral infections are well known to one skilled in the art.

It is believed that nucleoside analogs, once inside a proliferating cell, get phosphorylated (e.g., di- and triphosphate forms) and compete with the natural substrates (e.g., dGTP) of the telomerase reaction. The phosphorylated analogs can inhibit the incorporation of the natural substrates into the growing telomere DNA chain or can themselves become incorporated into DNA thereby interfering with telomerase or L1RT mediated polymerization activity, which eventually leads to termination of chain elongation. In essence, these nucleoside analogs, by termination of chain elongation, damage telomeric DNA, shorten telomeres and cause apoptosis. Damage to telomeres is more detrimental to rapidly growing (e.g., tumor) cells than to normal cells.

The acyclic nucleoside analogs of the present invention are more potent and selective inhibitors of telomere lengthening than the prior art known nucleoside analogs such as AZT; clinically acceptable doses are sufficient for realizing a decrease in telomere length and apoptosis or cell death of telomerase positive cells as compared to the nucleoside analogs such as AZT.

Other inhibitors of telomerase can be chemical agents such as 2,6-diamido-anthraquinones and carbocyanine dye, 3,3′-diethyloxadicarbocyanine (DODC) (and other telomeric DNA-interactive agents), a telomerase template antagonist (e.g., an antisense oligonucleotide covering the template region of the RNA in telomerase; specifically, for example, a lipid-conjugated thio-phosphoramidate, N3′-P5′, oligonucleotide sequence complementary to a template sequence contained with the RNA component of the RT), antisense constructs against telomerase RNA, sequence-specific peptide-nucleic acids directed against telomerase RNA. For purposes of the present invention, and notwithstanding the prior disclosures related to AZT as a telomerase inhibitor, AZT is not a telomerase inhibitor. In a preferred embodiment, the telomerase to be inhibited is a mammalian telomerase, such as a human telomerase.

Preferred inhibitors of L1RT are nucleoside analogs AZT, acyclovir ganciclovir, penciclovir and their pro-drugs. Preferred prodrugs are valacyclovir, valganciclovir and famciclovir. Other inhibitors of L1RT can be antisense constructs and oligonucleotides (see WO 2005/069880, the disclosure of which is incorporated by reference herein). For example, an antisense sequence corresponding to nucleotides 1987-2800 of human L1 reprotransposon (GenBank GI: 5070620) can be used. A sequence of nucleic acid residues or nucleotides that is a part of such a longer sequence of nucleic acid residues can be used as oligonucleotides. These include, for example, 5′-CCA GAG ATT CTG GTA TGT GGT GTC TTT GTT-3′, 5′-CTT TCT CTT GTA GGC ATT TAG TGC TAT AAA-3′,5′-CTC TTG CTT TTC TAG TTC TTT TAA TTG TGA-3′,5′-CTT CAG TTC TGC TCT GAT TTT AGT TAT TTC-3′, and 5′-TCC TGC TTT CTC TM TAG GCA-3′.

Preferred inhibitors of non-TERT and non-L1RT are nucleoside analogs AZT and ddI. Other inhibitors of non-TERT and non-L1RT can be antisense constructs and oligonucleotides. In a preferred embodiment, the non-L1RT to be inhibited is a human non-L1RT and/or a non-L1RT of retriviral origin.

Many of the inhibitors of the invention and methods for their manufacture have been previously disclosed. For example, the compound ddI is synthesized by the methods disclosed in the U.S. Pat. No. 5,011,774 and penciclovir is disclosed in the U.S. Pat. No. 5,075,445.

Preferred double cocktail is a combination AZT and ACV or PCV. Preferred triple cocktail is a combination AZT and acyclic nucleoside analogs and ddI, whereas the most preferred triple cocktail is AZT and ACV or prodrugs thereof and ddI. Preferred triple cocktail for the treatment of NSCLC (e.g., SK-LU-1 cells, which cells are believed to be both telomerase negative and L1RT negative) is AZT and PCV and ddI.

The combination of compounds described in the present invention inhibits reverse transcriptase(s) in cell extracts, in cultured cells and in vivo. Methods of inhibiting cancer cell growth using double cocktail of the present invention does not include the treatment of virus-associated cancers (e.g., Kaposi's sarcoma) wherein the occurrence of the cancer is linked with the infection by a virus chosen among Herpes viruses, Adenoviruses (21), Polyoma viruses, Papillomaviruses (HPV); Epstein-Barr viruses, Hepatitis DNA viruses (HBV or HCV).

In the present invention, the term “treat” or “treatment” means any treatment of a condition or disease involving proliferating cells, in particular inappropriately or pathologically proliferating cells or immortal cells in vitro, ex vivo or in a subject, or it means treatment of cancer. Bone marrow purging is an example of treatment ex vivo. The term includes inhibiting the condition or disease (for example, arresting its development) or relieving the condition or disease (for example, causing regression) or delaying the growth of proliferating cells or inducing apoptosis or programmed cell death. Some conditions intended to be treated by the method of the invention include benign (i.e., non-cancerous), pre-malignant and malignant (i.e., cancerous) tumors.

The term “an abnormal mammalian cell proliferation” is used herein, and it refers to a condition or disorder where a localized region of cells (e.g., a tumor) exhibit an abnormal (e.g., increased) rate of division as compared to their normal tissue counterparts. Conditions characterized by an abnormal mammalian cell proliferation, as used herein, include but are not limited to conditions involving solid tumor masses of benign, pre-malignant or malignant character. Indeed, normal cells sometimes become inappropriately or pathologically proliferating cells or immortal cells (e.g., due to p53 deficiency or mutations), and reproduce independently of cells' normal regulatory mechanisms. These cells are deemed to be inappropriately or pathologically proliferating cells or immortal cells because they deviate from the phenotype of normal cells as a result of activity of cellular elements, the RTs described above. Of course, the term “inappropriately proliferating cells” as used herein may be benign hyperproliferating cells but unless stated otherwise these cells refer to malignant hyperproliferating cells characteristic of a wide variety of tumors and cancers including stomach cancers, osteosarcoma, lung cancers, pancreatic cancers, adrenocortical carcinoma or melanoma, adipose cancers, breast cancers, ovarian cancers, cervical cancers, skin cancers, connective tissue cancers, uterine cancers, anogenital cancers, central nervous system cancers, retinal cancer, blood and lymphoid cancers, kidney cancers, bladder cancers, colon cancers and prostate cancers.

The term “Treating” or “treatment” of cancer in a subject or mammal or human includes one or more of the following: inducing apoptosis or inhibiting growth of the cancer, i.e., arresting its development, preventing spread of the cancer, i.e. preventing metastases, relieving the cancer, i.e., causing regression of the cancer, preventing recurrence of the cancer, and palliating symptoms of the cancer (e.g., amelioration of a adverse events of cytotoxic therapies by being able to suspend such therapies without the risk of significant cancer progression). The term “inhibiting cancer cell growth” or “inhibition of cell growth” may also mean reducing or preventing cell division.

Accordingly, in one aspect, the present invention is the discovery that a double cocktail or a triple cocktail, when administered to a subject in amounts that are effective in affecting telomere maintenance, can shorten the telomere length in a tumor. This aspect of the present invention includes interfering with telomere maintenance in a subject (or a patient), preferably a human, suffering from a telomere maintenance-mediated condition or disease.

Thus, in accordance with this aspect of the present invention there is provided a method of treating and a pharmaceutical composition for treating a telomere maintenance-mediated condition or disease. The treatment involves administering to a patient in need of such treatment a pharmaceutical composition comprising a pharmaceutical carrier and a therapeutically effective amount of each compound in the combination of the present invention, i.e., a therapeutically effective amount of a double cocktail or a triple cocktail.

With reference to double and triple cocktails, it should be noted that the combination of compounds of the instant invention can exist in any of the following forms as appropriate: (i) as individual compounds or components (e.g., at least three different tablets in case of triple cocktail) including forms wherein at least one of the individual components is in the form of a pharmaceutically acceptable salt, or (ii) individual compounds combined into one component (e.g., one tablet containing at least the three different inhibitors of triple cocktail) including a pharmaceutically acceptable salt of the combined compounds (i.e., a salt of the combination) or (iii) two different components in the case of triple cocktail including their pharmaceutical salts. Further, when practicing a method of the present invention, the individual components of the combination can be administered separately at different times during the course of therapy or concurrently in divided or single combination forms. For example, in a two-component combination (i.e., double cocktail), which is the inhibitor(s) of TERT and inhibitor(s) of L1RT, treatment with inhibitor(s) of L1RT can commence prior to, subsequent to or concurrent with commencement of treatment with inhibitor(s) of TERT. Likewise, treatment with triple cocktail combinations may be simultaneous, alternating or both simultaneous and alternating. The instant invention is therefore to be understood as embracing all such regimes of simultaneous or alternating treatment and the term “administering” or “administered” is to be interpreted accordingly.

Telomere maintenance affecting (or telomere shortening) combination of compounds means a combination of inhibitor(s) of TERT (also referred to as telomerase) and inhibitor(s) of L1RT (the combination referred to as double cocktail) or a combination of inhibitor(s) of telomerase RT, inhibitor(s) of L1RT and inhibitor(s) of non-L1RT (the combination referred to as triple cocktail). The therapy by administration of double cocktail or triple cocktail is referred to herein as background therapy.

As used herein, subject means a mammal including humans, nonhuman primates, dogs, cats, sheep, goats, horses, cows, pigs and other non-human mammals of veterinary interest. A “therapeutically effective amount” means that amount of a compound, a combination of compounds or compositions, double cocktail or triple cocktail which, when administered to a mammal, especially a human, for inducing apoptosis or treating or preventing a cancer, is sufficient to effect treatment for the cancer. “Effective amounts” are those amounts of a compound, a combination of compounds, double cocktail or triple cocktail, effective to reproducibly induce telomere shortening, G2 arrest and/or massive apoptosis in cancer cells in an assay in comparison to levels in untreated cells. An “effective amount” also means as an amount of a compound, a combination of compounds, double cocktail or triple cocktail, that will decrease, reduce, inhibit or otherwise abrogate the growth of a cancer cell.

In another aspect, the present invention concerns methods for inhibiting pathologically proliferating cells e.g., tumor cells, by contacting the cells with a double or triple cocktail. In general, the methods include a step of contacting a pathologically proliferating cell (e.g., a cancer cell) with an amount of a double or triple cocktail which is effective to reduce or inhibit the proliferation of the cell, or induce programmed cell death. The present methods can be performed on cells in culture, e.g., in vitro or ex vivo, or can be performed on cells present in a subject, e.g., as part of an in vivo therapeutic protocol. The therapeutic regimen can be carried out on a human or on other animal subjects in need of such a therapy. The therapeutic specificity of the background therapy disclosed in the present invention represents a promising alternative to conventional highly toxic regimens of cytotoxic anticancer agents, such as conventional cytotoxic chemotherapy or even DNA damaging therapy.

From the above, one skilled in the art may readily appreciate that the combination of inhibitors and methods of the invention in certain instances may be useful for replacing existing surgical procedures or drug therapies, although in most instances the present invention is useful in improving the efficacy and/or ameliorating the toxic effects of the existing therapies for treating such conditions. Specifically, the use of combination of inhibitors in the methods of the present invention can improve the efficacy and/or ameliorating the toxic effects of the existing therapies by selectively sensitizing or increasing the sensitivity of the abnormally proliferating cells (e.g., tumor cells) to various DNA damaging agents.

Accordingly the background therapy described herein may be combined with other anticancer therapies and used to treat the subjects. For example, a selected background therapy may be administered to a subject in combination with another anti-proliferative (e.g., an anti-cancer) therapy. As used herein, “in combination with another anti-proliferative therapy or therapies” means that the background therapy may be administered prior to, during or after the other anti-proliferative therapy or therapies. Suitable anti-cancer therapies include surgical procedures to remove the tumor mass or DNA damaging therapy (described more fully below) including localized radiation. Thus, the other anti-proliferative therapy may be administered before, concurrent with, or after treatment with the combination of inhibitors of the present invention. There may also be a delay of several hours, days and in some instances weeks between administration of the different treatments, such that the background therapy may be administered before or after the other treatment.

As an example, the background therapy may be administered in combination with surgery to remove an abnormal proliferative cell mass. Surgical methods for treating gastro-intestinal tumor conditions include intra-abdominal surgeries such as total colectomy and gastrectomy. In these embodiments, the compounds of background therapy may be administered either by continuous infusion or in a single bolus. Treatment, during or immediately after surgery, may involve a lavage, soak or perfusion of the tumor excision site with a pharmaceutical preparation of the inhibitors in a pharmaceutically acceptable carrier. In some embodiments, the combination of inhibitors is administered at the time of surgery as well as following surgery in order to inhibit the formation and development of metastatic lesions. The administration of the agent may continue for several hours, several dais, several weeks, or in some instances, several months following a surgical procedure to remove a tumor mass.

As already described, while the background therapy can be utilized alone, this therapy may also be combined with a DNA damaging treatment or therapy for a therapeutic effect that is greater than expected for the DNA damaging treatment or therapy alone. This is because the cells arrested at G2/M phase of the cell cycle are generally very sensitive to DNA damaging treatment or therapy. DNA damaging treatments or therapy includes genotoxic chemotherapy (with genotoxic drugs), radiation therapy (with gamma-irradiation, X-rays, radioisotopes and the like) and/or photodynamic therapy (e.g., with 5-aminolevulinic acid). Agents that damage DNA are well known to one skilled in the art and are widely used in a clinical setting for the treatment of neoplasms. For instance, genotoxic drugs are chemotherapy agents that affect nucleic acids and alter their function. These drugs may directly bind to DNA or they may indirectly lead to DNA damage by affecting enzymes involved in DNA replication. Rapidly dividing cells are particularly sensitive to genotoxic agents because they are actively synthesizing new DNA. If enough damage is done to the DNA of a cell it will often undergo apoptosis, the equivalent of cellular suicide. The genotoxic chemotherapy treatments include: (1) alkylating agents: the first class of chemotherapy agents used. These drugs modify the bases of DNA, interfering with DNA replication and transcription and leading to mutations; (2) intercalating agents: these drugs wedge themselves into the spaces between the nucleotides in the DNA double helix. They interfere with transcription, replication and induce mutations; and (3) enzyme inhibitors: these drugs inhibit key enzymes, such as topoisomerases, involved in DNA replication inducing DNA damage.

A number of such agents have been developed, particularly useful are agents that have undergone extensive clinical testing and are readily available. The agent 5-fluorouracil (5-FU) (or related agent capecitabine) is one such agent that is preferentially used by neoplastic tissue, making it particularly useful for targeting neoplastic cells. Thus, although quite toxic, 5-FU, is applicable with a wide range of carriers, including topical and even intravenous administrations. Platinum compound cisplatin has also been widely used to treat cancer, with efficacious doses used in clinical applications of 20 mg/m2 for 5 days every three weeks for a total of three courses. cisplatin is not absorbed orally and must therefore be delivered via injection intravenously, subcutaneously, intratumorally or intraperitoneally. Other DNA damaging agents contemplated to be of use in the present invention include capecitabine (Xeloda®), cyclophosphamide, oxaliplatin, busulfan, carboplatin, carmustine, chlorambucil, doxorubicin, daunorubicin, epirubicin, etoposide, idarubicin, temozolomide, ifosfamide, lomustine, dacarbazine, mechlorethamine, melphalan, mitomycin C, mitoxantrone, irinotecan, and topotecan and the like.

These drugs are used to treat a variety of solid cancers and cancers of blood cells. The goal of treatment with any of these agents is the induction of DNA damage in the cancer cells. DNA damage, if severe enough, will induce cells to undergo apoptosis, the equivalent of cellular suicide. However, the DNA damaging agents affect both normal and cancerous cells. The selectivity of the drug action is based on the sensitivity of rapidly dividing cells, such as cancer cells, to treatments that damage DNA The mode of action also explains many of the side effects of treatment with these drugs. But, dividing non-cancerous cells, such as those that line the intestine or the stem cells in bone marrow, also are often killed-along with the cancer cells as a result of the non-specific interaction of the drug (which may be direct or indirect interaction) with the DNA of the non-cancerous cells. In addition to being cytotoxic (cell poisons), these drugs are also mutagenic (cause mutations) and carcinogenic (cause cancer). Treatment with these drugs carries with it the risk of secondary cancers, such as leukemia perhaps due to the development of resistance to the underlying DNA damaging therapy. Furthermore, cancerous cells exposed to slightly sub-lethal concentrations of a chemotherapeutic agent will very often develop resistance to such an agent, and quite often cross-resistance to several other genotoxic agents as well. Subjects resistant to a given genotoxic therapy often have very short survival times due to uncontrolled growth of resistant cells.

As already described above, the uncontrolled growth or proliferation through telomere maintenance by TERT and/or other RTs is a hallmark of cancer cells. Because the compounds or the combinations of compounds used in background therapy affect telomere maintenance, the double or triple cocktails should selectively damage telomeric DNA and induce progressive telomere shortening only in uncontrollably proliferating cells or cancer cells. Because of this selectivity of the double or triple cocktails, a wide therapeutic index relative to their toxicity towards non-malignant cells can be realized. In contrast, induction of DNA damage by the agents used in DNA damaging therapy is not limited to telomeric DNA at the outset and is thus non specific. In that regard, the DNA damage from the agents used in the DNA damaging therapy is broader and, as described above, is associated with many side effects and the risk of developing leukemia.

It is known in the art that leukemia is a cancer of bone marrow (which is a factory for all different types of blood cells; red blood cells, white blood cells and platelets) and blood. In particular, leukemia is a malignant cancer and is characterized by the uncontrolled proliferation of blood cells (e.g., white blood cells). It is believed that the double and triple cocktails would be invaluable players and/or allies in the war on leukemia, whether the cancer develops as a primary cancer or secondary cancer. The cocktails should retain their efficacy against all malignant cells (leukemic and non-leukemic cells) by damaging telomeric DNA and inducing G2 arrest even after prolonged exposure to the cocktails. In other words, the malignant cells that could otherwise escape due to the development of resistance to the underlying DNA damaging agents, remain vulnerable to telomeric DNA damage and G2 arrest and eventually their death in the presence of double or triple cocktails.

Accordingly, in another aspect of the invention, a method of sensitizing tumor cells to a DNA damaging therapy is provided. This method involves administration of a sensitizing effective amount of a combination of double cocktail or triple cocktail and sensitize tumor cells prior to or during DNA damaging therapy. A sensitizing effective amount is that amount effective to induce G2/M phase arrest. Preferably, the background therapy is administered first to expose tumor cells to a double cocktail or triple cocktail for the duration of at least several cycles of proliferation, and more preferably at least 14 days of proliferation. Then the DNA damaging therapy is administered in addition to the background therapy for a certain duration (e.g., until the DNA damaging therapy manifests clinically unfavorable toxic effects or just prior to that stage) and the DNA damaging therapy is suspended or withdrawn depending on the treating physician's assessment. The administration of the background therapy may precede at least one aspect of the DNA damaging therapy (such as the administration of one dose of a genotoxic chemotherapeutic agent, biologic therapy agent, or radiation therapy) by as little as a few minutes (for example, during the same day or during the same treatment visit) to as much as several weeks, for example from one to five weeks, e.g. one to three weeks.

An alternative preferred method is the administration of background therapy during the administration of a DNA damaging regimen. This will certainly be the case in situations where DNA damaging therapy is already underway but one desires background therapy to induce G2/M phase arrest and thereby sensitize the pathologically proliferating cells (e.g., tumor cells).

In either case, it is preferred that a selected background therapy is continued after a DNA damaging regimen is terminated, and it is continued for at least several weeks, months, years, or longer. In a clinical setting, the background therapy would essentially allow a treating physician to effectively combat the patient's cancer if it reappears or proves to be refractory to other therapies. This is especially so for a cocktail combination of the present invention of minimal or no toxicity to the patient, as it provides for a favorable risk-to-benefit ratio. For example, the double and triple cocktail of nucleoside analogs (AZT and ACV or AZT, ACV/PCV and ddI) at the low amounts needed to inhibit RTs, are minimally or not toxic to the patient, and provide such favorable risk-to-benefit ratio.

The above strategy of continuous background therapy interspersed with DNA damaging therapy on a subject (e.g., human) would allow not only for shorter durations of DNA damaging therapy but also for the administration of lower doses of the clinically approved cytotoxic agents thus, reducing the induction of adverse events in the subject, such as a human cancer patient. For example, clinically relevant adverse events of capecitabine (Xeloda®) monotherapy are well known in the art. These include hand-and-foot syndrome, cardiotoxicity, stomatitis, diarrhea, nausea, vomiting, neutropenia, electrolyte imbalance neurotoxicity and hyperbilirubinemia. In the presence of background therapy (which can potentially induce programmed cell death through G2/M phase arrest), however, one may be able to reduce the originally prescribed therapeutic doses of such cytotoxic agents and enhance their efficacy on the sensitized cells, thereby in essence providing for amelioration therapy. As a net result, amelioration of toxicities from DNA damaging therapy (simply by being able to suspend or reduce toxic doses of the cytotoxic agents), enhanced tumor shrinkage, delayed tumor growth and/or elimination of cancer and extended survival time in human cancer patients are expected following the administration of background therapy. Of course, as already mentioned above, surgical excision of tumors in addition to or in place of non-surgical therapies may also be carried out.

The drug cocktail can also be used for preventing the disease from occurring in an animal which may be predisposed to the disease but does not yet experience or display symptoms of the disease or can be used for reducing the incidence of cancer. Accordingly, in yet another aspect, the present invention discloses a method of preventing cancer in a patient by identifying a patient prone to have cancer and/or harboring cells capable of becoming malignant, both of which are difficult to detect by conventional means (physical examination). The method involves administering a background therapy to the patient such that prevention of cancer development is achieved. As examples, some of the conventional methods to detect tumors are physical methods (e.g., palpation), pathological methods (e.g., blood in urine or stool), or imaging methods (e.g., X-ray, CAT scan, PET scan, ultrasound sonogram). Identification of patients that are likely to have a cancer or harboring an undetectable cancer cells can also be achieved by monitoring biomarkers or genetic defects.

For example, it is known in the art that loss of wild-type p53 (wt-p53) function generally leads to uncontrolled cell cycling and replication, inefficient DNA repair, selective growth advantage and, hence, tumor formation. In fact, it has been reported that the p53 gene is mutated in more than 50% of tumors. As another example, a female patient, not currently having a detectable tumor, could have a mutation in the BRCA1 or BRCA2 gene, showing a strong predisposition for the development of a breast or ovarian cancer. Similarly, biomarkers, tumor suppressor oncogene protein 53, oncogene c-erbB-2 and combinations thereof for breast carcinoma have been found in salivary secretion. Another example, where molecular markers have been reported is lung cancer. Lung cancer includes small cell lung carcinomas and non-small cell lung cancer (NSCLC) (adenocarcinomas, squamous cell lung carcinomas, and large cell carcinomas) is one of the leading causes of cancer death in the world. The NSCLC accounts for nearly 80% of lung malignant tumors and it is associated with a poor prognosis. It is known in the art that lung cancer is the result of molecular changes in the cell, resulting in the deregulation of pathways which control normal cellular growth, differentiation, and apoptosis. Various genes such as proto-oncogenes and tumor suppressor genes are found to be mutated or have abnormal expression patterns in this disease. Indeed, a set of molecular signatures modulated by Rb2/p130 in lung cancer cells has been reported. The molecular signatures include expression products of one or more of the following genes: B-MYB, PCSK7, STK15, ELK1, NOL1, MAGEA3/6, PIM1, CCND1, CDR2, and RAF1, all of which can be modulated by RB2/p130. Thus, the art identified molecular signatures or markers in various tissue samples may provide a basis for assessing the likelihood of tumor occurrence in patients. Treatment of such patients with a given background therapy described above with or without cytotoxic agent(s) would be effective to prevent cancer.

This invention encompasses the use of telomerase inhibitor-based cancer therapy for a wide variety of tumors and cancers affecting skin, connective tissues, adipose, breast, lung small cell lung carcinomas and non-small cell lung cancer (NSCLC)), stomach (gastric cancer), pancreas, ovary, cervix, uterus, kidney, bladder, colon, prostate, anogenital, central nervous system (CNS), retina and blood and lymph (lymphomas resulting from the expression of CDK9/CYCLIN T1 in precursor T cells, precursor B cells, germinal center cells, activated T cells or Reed-Sternberg cells) and a number of other cancers mentioned elsewhere in this disclosure.

While it is possible for the compound(s) for use in the above-indicated utilities and indications to be administered alone, it is preferable to present them as pharmaceutical formulations. Particularly in some situations, where clinical applications are contemplated pursuant to regulatory guidelines, it may be necessary to prepare pharmaceutical compositions or formulations of drugs in a form appropriate for the intended application. Generally, this will entail preparing compositions that are essentially free of pyrogens, as well as other impurities that could be harmful to humans or animals. The inhibitors for use in the present invention may be dissolved in water (preferably sterile drinking water) or pharmaceutically or pharmacologically acceptable carrier. The term “pharmaceutically or pharmacologically acceptable” refer to carriers and compositions that do not produce adverse, allergic, or other untoward reactions when administered to an animal or a human. It includes any and all solvents, dispersion media, coatings, antibacterial and antifungal agents, isotonic and absorption delaying agents and the like. The use of such media and agents for pharmaceutically active substances is well known in the art.

In a double cocktail combination, as described above, there should be at least one inhibitor of telomerase and an inhibitor of L1RT that is different from the inhibitor of telomerase. The inhibitors can be either together in a single composition or pharmacological formulation or separately in two distinct compositions or formulations. In a triple cocktail combination, there should be at least one inhibitor of telomerase, an inhibitor of L1RT that is different from the inhibitor of telomerase and an inhibitor of non-L1RT that is different from the inhibitors of telomerase and L1RT. These inhibitors also can be either together in a single composition or formulation, or be in three distinct compositions or formulations.

These pharmaceutical compositions may be in any suitable form including the form of orally-administrable suspensions or tablets; nasal sprays; sterile injectable preparations, for example, as sterile injectable aqueous or oleaginous suspensions or suppositories.

Administration of the compounds and compositions according to the present invention will be via any common and suitable route so long as the target tissue is available via that route. This includes oral, nasal, buccal or topical. Alternatively, administration may be by orthotopic, intradermal, subcutaneous, intramuscular, intraperitoneal or intravenous injection. In some embodiments, the compounds or compositions may be administered in a systemic manner, via administration routes such as, but not limited to, oral, intravenous, intramuscular and intraperitoneal administration. Systemic administration routes may be preferred, for example, if the subject is known to have or is suspected of having metastatic lesions. In this way, all tumor Sites, whether primary or secondary, may receive the agent.

In other embodiments, the compounds or compositions are administered in the form of sustained release formulations so that repeated administration and inconvenience to the patient may be avoided. Various forms of sustained release microspheres or microcapsules are also available or are being developed as delivery systems for the rapidly expanding class of peptide and non-peptide therapeutic or pharmacological agents. For example, sustained release microspheres or microcapsules are, known in the administration of antitumoral drugs, peptides, and simple basic compounds such as thioridazine and ketotifen where using the biodegradable polymer materials. Further, for example, in recent years, a variety of injectable depot formulations in which therapeutic drugs encapsulated in, and released slowly from, microspheres made of biodegradable polymers have been reported (U.S. Pat. Nos. 5,478,564, 5,540,973, 5,609,886, 5,876,761, 5,688,530, 5,631,020, 5,631,021 and 5,716,640). Indeed, long acting injectable depot formulations of GnRH analogues (agonists and antagonists) are being used and/or tested for the treatment of various pathological and physiological conditions in mammals, particularly in humans (Kostanski et al., 2001, BMC Cancer, 1:18-24). The treatments are for, among other things, the management of sex hormone-dependent diseases such as prostate cancer and endometriosis and for the control of male fertility. Thus, steady release of the compounds or cocktails described in the present invention can be implemented in animals by using compositions containing biodegradable biocompatible polymers. One obvious goal behind in all of these polymeric compositions is that the biologically active agent (e.g., a peptide or protein) of interest can be administered less frequently, sometimes at lower overall doses, than when formulated as a solution without the use of polymers in them. Use of a long-term sustained release formulations or implants may be particularly suitable for treatment of chronic conditions, such as the suspected presence of dormant metastases. Long-term release, as used herein, means that the formulation or implant is made and arranged to deliver therapeutic levels of a double or triple cocktail described above for at least 30 days, at least 60 days and more preferably for several months.

In administering the compounds of the invention to a subject, dosing amounts, dosing schedules, routes of administration and the like may be selected so as to affect the other known activities of these compounds. For example, amounts, dosing schedules and routes of administration can be selected as described herein, whereby therapeutically effective levels for inhibiting cell proliferation are provided.

According to some embodiments of the invention, however, the compounds or compositions are administered locally. In some embodiments, the compounds or compositions are targeted to a tumor. This can be achieved by the particular mode of administration. For example, easily accessible tumors such as breast or prostate tumors may be targeted by direct needle injection to the site of the lesion. Lung tumors may be targeted by the use of inhalation as a route of administration. Inhalation may be used in either systemic or local delivery. A preferred route is direct intra-tumoral injection, injection into the tumor vasculature or local or regional administration relative to the tumor site.

The compounds used in the methods of this invention can be administered to mammals (e.g., humans) in the dosage ranges specific for each compound. When antisense oligonucleotides are used as inhibitors of RTs, these may be administered at a dose of 5-50 μM, preferably 30 μM in the case of 2′-o-methyl RNA or 10 μM antisense oligonucleotide (Pitts et al., Inhibition of human telomerase by 2′-m-methyl-RNA, Proc. Natl. Acad. Sci. USA, 95: 11549-11554, 1998), 10 μM phosphorothioate oligonucleotide or 10 μM of a hexameric phosphorothioate oligonucleotide in which a pair of three bases are separated by a nine-carbon phosphoramidite spacer (Mata et al., A hexameric phosphorothioate oligonucleotide telomerase inhibitor arrests growth of Burkitt's lymphoma cells in vitro and in vivo. Toxicol. Appl. Pharmacol., 144:189-197, 1997; Page et al., The cytotoxic effects of single-stranded telomere mimics on OMA-BL1 cells. Exp. Cell Res., 252: 41-49, 1999). Small molecule inhibitors RT such as diaminoanthraquinone derivates may be used, for example, at 10 mM (Perry et al., 1998, 1,4- and 2,6-Disubstituted amidoanthracene-9,10-dione derivatives as inhibitors of human telomerase. J. Med. Chem., 41: 3253-3260).

For example, when nucleoside analogs are used as inhibitors of RTs, a nucleoside analog or a pharmaceutically acceptable salt thereof, may be administered by any suitable route (e.g., orally or parenterally) in a dosage range between about 10 mg and about 4000 mg per day, divided into between one and four doses per day. One preferred dosage range is between about 200 mg and about 1200 mg every 8 hours.

In general, however, a suitable effective dose will be in the range 0.1 to 250 mg per kilogram bodyweight of recipient per day, preferably in the range 1 to 100 mg per kilogram bodyweight per day and most preferably in the range 5 to 20 mg per kilogram bodyweight per day; an optimum dose is about 10 mg per kilogram bodyweight per day. The desired dose is preferably presented as two, three, four or more sub-doses administered at appropriate intervals throughout the day. These sub-doses may be administered in unit dosage forms, for example, containing 10 to 1000 mg.

For example, one preferred dosage range of acyclovir or its prodrug, Valtrex®, is 100 to 400 mg of active ingredient per unit dosage form. It is well understood by those skilled in the art that different dosage forms of the prodrugs may command different dosage ranges usually established by determining the blood level concentrations of the metabolite (e.g., acyclovir if the prodrug is Valtrex®).

Similarly, for an oral formulation of ganciclovir or its prodrug Valcyte®, for example, a therapeutically effective amount may vary from about 1 to 250 mg per Kg body weight per day, preferably about 7 to 100 mg/Kg body weight per day. Thus, for a 70 Kg human, a therapeutically effective amount is from about 70 mg/day to about 7 g/day, preferably about 500 mg/day to about 5 g/day. The effective dose of penciclovir and its prodrug, Famvir, can in general be in the range of from 1.0 to 20 mg/kg of body weight per day or more usually 2.0 to 10 mg/kg per day. One preferred dosage range of AZT (zidovudine) is between about 50 mg and about 600 mg every 8 hours. One preferred dosage range of ddI is between about 10 mg and about 500 mg twice daily. The dosages for genotoxic chemotherapeutic agents can be those recommended by manufacturers for a given disease therapy.

Further details on dosage and administration of various inhibitors of the RTs described above (including acyclovir, Valtrex®, ganciclovir, Valcyte®, Famvir, Retrovir®-AZT or zidovudine- and Videx, Xeloda®, Cisplatin, etc.) can be found in Physician's Desk Reference, PDR, 58th Edition, 2004, the contents of which are incorporated herein by reference.

The present invention also encompasses the use of various animal models. By developing or isolating cell lines that express telomerase one can generate disease models in various laboratory animals. These models may employ the subcutaneous, orthotopic or systemic administration of cells to mimic various disease states. For example, the HeLa cell line can be injected subcutaneously into nude mice to obtain telomersse positive tumors. The resulting tumors should show telomerase activity in telomeric repeat amplification protocol (TRAP) assay. Such animal models provide a useful vehicle for testing the nucleoside analogs individually and in combinations as well.

In general, the level of telomerase activity or L1RT activity in a cell can be measured as described, for example, in the Applicant's U.S. Patent Application 60/655,105, entitled “Modulation Of Telomere Length In Telomerase Positive Cells For Cancer Therapy” filed Mar. 25, 2005 and the International Patent Application PCT/US05/001319 entitled “Modulation Of Line-1 Reverse Transcriptase” filed Jan. 18, 2005, which patent applications are incorporated herein by reference. The level of telomerase activity (or L1RT) activity in a cell may also be measured by any other existing method or equivalent method. By “elevated level” of telomerase activity or L1RT activity, it is meant that the absolute level of telomerase activity or L1RT activity in the particular cell is elevated compared to normal cells in that subject or individual, or compared to normal cells in other subjects or individuals not suffering from the condition. Examples of such conditions include cancerous conditions, or conditions associated with the presence of cells which are not normally present in that individual.

Determining the effectiveness of a compound in vivo may involve a variety of different criteria including, but are not limited to, survival, tumor regression, arrest or slowing of tumor progression, elimination of tumors and inhibition or prevention of metastasis.

Treatment of animals with a test compound would involve the administration of the compound or composition in an appropriate form to the animal. The pharmaceutical compositions, inhibitory or antagonistic agents of the present invention can be administered in a variety of ways including but not limited to oral, parenteral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

The acyclic nucleoside analogs of the present invention and/or compositions of the present invention (which compositions can include the prior art known acyclic and non-acyclic nucleoside analogs) would be important in a number of aspects. They would be useful as selective inhibitors and for applying selective pressure on cells to switch mechanisms of telomere elongation. They would be important in regimens for the treatment of telomerase/L1RT-related cancers, whether administered alone or in combination with chemo- and/or radiotherapeutic regimens known to one skilled in the art in the treatment of cancer. Alternatively, by simply reducing telomerase or L1RT activity, these compositions will be instrumental in selectively inducing massive apoptosis of cancer cells.

The nucleoside analogs may be administered in a physiologically or pharmaceutically acceptable carrier to a host for treatment of proliferative diseases, etc. Pharmaceutically acceptable carriers are determined in part by the particular composition being administered as well as by the particular method used to administer the composition.

In an aspect of the present invention, methods for preventing or treating disorders caused by the presence of inappropriately or pathologically proliferating cells or immortal cells in mammals are provided. The inappropriately or pathologically proliferating cells or immortal cells exist and reproduce independently of cells' normal regulatory mechanisms. These cells are pathologic because they deviate from normal cells as a result of activity of a cellular element, i.e., telomerase. Of course, the inappropriately proliferating cells as used herein may be benign hyperproliferating cells but, unless stated otherwise, these cells refer to malignant hyperproliferating cells such as cancer cells of all kinds including, for example, osteosarcoma, breast carcinoma, ovarian carcinoma, lung carcinoma, adrenocortical carcinoma or melanoma. In one embodiment of the invention, post-transplant lymphoproliferative disease (PTLD), which is a cancer of the blood, is excluded from the scope of cancers contemplated herein.

In particular, methods for preventing or treating human tumors characterized as expressing telomerase are provided. The prevention or treatment of the disorders, according to the present invention, is achieved by the utilization of acyclic nucleoside analogs (inhibitors or antagonists of telomerase) of the present invention. In an embodiment of the invention, the inhibitor(s) or antagonist(s) used in the present invention are those acyclic nucleoside analogs that directly interact with telomerase responsible for telomere elongation to inhibit its activity and/or those that get incorporated into telomere and thus prevent telomere from further elongation despite the functional telomerase thereby inhibiting the growth of cells expressing telomerase. Thus, the inhibitors or antagonists of telomerase are used for inhibiting the growth of cells. For example, when the inhibitors or antagonists of telomerase are administered to a patient, these cause progressive telomere shortening, cell cycle arrest in the cells and/or massive apoptosis of cells expressing telomerase. In the present invention, the terms “inhibiting the growth” or “inhibition of growth” may also mean reducing or preventing cell division. Inhibition of growth of cells expressing telomerase, according to the present invention, may be about 100% or less but not 0%. For example, the inhibition may be from about 10% to about 100%, preferably at least about 25%, and more preferably at least about 50%, still more preferably at least about 90%, 95% or exactly 100% compared to that of the control cells (control cells express telomerase but are not treated with an inhibitor or antagonist). The inhibition of growth can be measured by any methods known in the art. For example, viable cell number in treated samples can be compared with viable cell number in control samples, determined after incubation with vital stains. In addition, growth inhibition can be measured by assays that can detect reductions in cell proliferation in vitro or in vivo, such as tritiated hydrogen incorporation assays, BdU incorporation assay, MTT assay, changes in ability to form foci, anchorage dependence or losing immortalization, losing tumor specific markers, and/or inability to form or suppress tumors when injected into animal hosts (Dorafshar et al., 2003, J Surg Res., 114:179-186; Yang et al., 2004, Acta Pharmacol Sin., 25:68-75).

The development of a cancerous tumor from a single immortalized cell or few such cells may take several months to years in humans. By practising the present invention, however, cancer can be prevented because the ability of the tumorigenic cells treated with compositions containing one or more acyclic nucleoside analogs lose their proliferative potential before they have had a chance to grow into a tumor. Further, periodic preventative administration of the inhibitors or antagonists to at risk groups in order to stop tumor progression before clinical-manifestation of cancer could potentially decrease the rate of new cancer cases significantly.

The nucleoside compounds may be administered either singly or in combinations of different analogs and by any routes of administration, including oral administration. The acyclic nucleoside analogs SN 1, SN 2 are the preferred nucleoside analogs and SN 1 is the most preferred one. Among the prior art known acyclic nucleoside analogs, ACV, GCV or their L-valil esters valganciclovir (V GCV) and valacyclovir (V-ACV) are the preferred nucleoside analogs. All of them are commercially available and the formulations are described in a number of patents and publications.

The cells with telomerase and/or L1RT activity should be selectively targeted because these cells depend on telomerase and/or L1RT for elongating or maintaining telomeres and the elongation or maintenance of telomeres requires the interaction of the nucleosides and/or their analogs with telomerase or L1RT. To the extent any specific targeting agent is desired for delivering the analogs to exert anti-cancer effects, the use of targeted compounds of the formulas (I) to (VI), PCV or ACV or GCV and/or other analogs are contemplated herein. Accordingly, in some embodiments, pharmaceutical compositions may have the active compound, in this case, any of compounds of the formulas (I) to (VI), PCV, ACV and GCV, which has been conjugated to a targeting agent (e.g., a peptide) for specific delivery to particular target cells or to nuclear portion within cells.

The dose of a given inhibitor or antagonist of telomerase and L1RT can be determined by one of ordinary skill in the art upon conducting routine experiments. Prior to administration to patients, the efficacy may be shown in standard experimental animal models. In this regard any animal model for telomerase induced cancer known in the art can be used (Hahn et al., 1999, Nature Medicine, 5(10):1164-1170; Yeager et al., 1999, Cancer Research, 59(17): 4175-4179). The subject, or patient, to be treated using the methods of the invention is preferably human, and can be a fetus, child, or adult. Other mammals that may be treated can be mice, rats, rabbits, monkeys and pigs.

The acyclic nucleoside analogs, inhibitors or antagonists of the present invention can be used alone or in combination with other chemotherapeutics. For example, therapy of telomerase induced cancers may be combined with chemo and/or radiotherapy to treat cancers induced by telomerase or some other factors. Examples of chemotherapeutic agents known to one skilled in the art include, but are not limited to, anticancer drugs such as bleomycin, mitomycin, nitrogen mustard, chlorambucil, 5-fluorouracil (5-FU), floxuridine (5-FUdR), methotrexate (MTX), colchicine and diethylstilbestrol (DES). To practice combined therapy, one would simply administer to an animal an inhibitor component of the present invention in combination with another anti-cancer agent (chemo or radiation) in a manner effective to result in their combined anti-cancer actions within the animal or patient. The agents would therefore be provided in amounts effective and for periods of time effective to result in their combined presence in the region of target cells. To achieve this goal, the agents may be administered simultaneously, and in the case of chemotherapeutic agents, either in a single composition or as two distinct compositions using different administration routes. Alternatively, the two treatments may precede, or follow, each other by, e.g., intervals ranging from minutes to hours or days. By way of example, and not limitation, the average daily doses of GCV for systemic use may be 100 mg/kg per day for human adults, 50 mg/kg per day for mice and human infants.

Some variation in dosage may occur depending on the condition of the subject being treated. The physician responsible for administration will be able to determine the appropriate dose for the individual patient and may depend on multiple factors, such as, the age, condition, file history, etc., of the patient in question.

Accordingly, the methods of the invention can be used in therapeutic applications for conditions and diseases associated with telomerase induced pathological proliferation of cells. Diseases that would benefit from the therapeutic applications of this invention include all diseases characterized by cell hyperproliferation including, for example, solid tumors and leukemias, and non-cancer conditions. It is further contemplated that the method of the invention can be used to inhibit the growth of cancer cells not only in an in vivo context but also in an ex vivo situation. The method of the invention is particularly useful for inhibiting the growth of pathologically proliferating human cells ex vivo, including, but not limited to, human cancer cells—osteosarcoma, breast carcinoma, ovarian carcinoma, lung carcinoma, adrenocortical carcinoma or melanoma.

The present invention provides methods and kits for identifying inappropriately, pathologically or abnormally proliferating cells due to the expression of telomerase in the cells. The methods can be used as a screening method that aids in diagnosing the presence of a cancerous cell or tumor in a patient by determining the presence (and/or level) of expression of telomerase in tissues from the patient, the presence of telomerase expression at elevated levels is being indicative of cancer cells or pathological cell proliferation in the patient.

For example, cancerous tumor samples can be diagnosed by their inability to proliferate in the presence of the acylic nucleoside analogs of the present invention. The diagnosis may further involve the detection of telomerase specific mRNA expression measured by a variety of methods including, but not limited to, hybridization using nucleic acid, Northern blotting, in situ hybridization, RNA microarrays, RNA protection assay, RT-PCR, real time RT-PCR, or the presence of telomerase catalytic subunit encoded protein measured by variety of methods including, but not limited to, Western blotting, immunoprecipitation or immunohistochemistry, or enzymatic activity of telomerase (TRAP assay and its modifications^{4,26,27}).

In a preferred embodiment, nucleic acid probes directed against telomerase catalytic subunit RNA can be used to detect presence and/or increases in telomerase catalytic subunit RNA mRNA levels in tissues undergoing rapid proliferation, such as primary cancer cells, including human osteosarcoma, breast carcinoma, ovarian carcinoma, lung carcinoma, adrenocortical carcinoma or melanoma. Thus, the present invention provides methods of using nucleic acid probes that are complementary to a subsequence of an telomerase to detect and identify pathologically proliferating cells, including cancer cells. For example, the method for identifying a pathologically proliferating cell may involve using a nucleic acid probe directed against hTERT mRNA or L1RT mRNA to compare the level of expression of hTERT mRNA or L1RT mRNA in a test cell with the level of expression of hTERT mRNA or L1RT mRNA in a control cell. A test cell is identified as a pathologically proliferating cell when the level of hTERT or L1RT expression is observed as in the control cell. The nucleic acid probe used in the method of the invention, however, may also be substantially complementary to an hTERT mRNA or L1RT mRNA sequence of human, mouse or other mammal.

It will be apparent to one of ordinary skill in the art that substitutions may be made in the nucleic acid probe which will not affect the ability of the probe to effectively detect the hTERT mRNA or L1RT mRNA in pathologically proliferating cells (e.g., cancer cells) and thus, such substitutions are within the scope of the present invention. The nucleic acid probe used in the method of the present invention can be a DNA probe, or a modified probe such a peptide nucleic acid probe, a phosphorothioate probe, or a 2′-O methyl probe. The length of the nucleic acid probe may be from about 8 or 10 to 50 nucleotides, preferably from about 15 to 25 nucleotides in length. The method of the invention can be readily performed in a cell extract, cultured cell, or tissue sample from a human, a mammal, or other vertebrate.

The methods of the present invention are useful for detecting the inappropriately, pathologically or abnormally proliferating cells due to the expression of telomerase in the cells in vitro, in cell cultures, and in human cells and tissues, such as solid tumors and cancers (e.g., human osteosarcoma, breast carcinoma, ovarian carcinoma, lung carcinoma, adrenocortical carcinoma or melanoma).

The present invention also provides kits for detecting and/or inhibiting hyperproliferating cells or cancer cells. The kit can have compounds of the formulas (I) to (VI), and optionally PCV, ACV, GCV, valganciclovir valacyclovir or other acyclic nucleoside analogs and/or have a nucleic acid probe that is fully or substantially complementary to a subsequence of an hTERT mRNA or L1RT mRNA.

The pharmaceutical compositions, inhibitory or antagonistic agents of the present invention can be administered in a variety of ways including orally, topically, parenterally e.g. subcutaneously, intraperitonealy, by viral infection, intravascularly, etc. Depending upon the manner of introduction, the compounds may be formulated in a variety of ways. Formulations suitable for oral administration can be liquid solutions. Formulations suitable for parenteral administration (e.g., by Intraarticular, intraventricular, intranasal, intravenous, intramuscular, intradermal, intraperitoneal, and subcutaneous routes) include aqueous and non-aqueous, isotonic sterile injection solutions. In the practice of this invention, compositions can be administered, for example, by intravenous infusion, orally, topically, parenterally or intraperitoneally. Oral and parenteral administrations are the preferred methods of administration. Techniques for formulation and administration are routine in the art and further details may be found, for example, in Remington's Pharmaceutical Sciences (2000), Gennaro AR(ed), 20th edition, Maack Publishing Company, Easton, Pa.

Therapeutically effective amount or pharmacologically effective amount are well recognized phrases in the art and refer to that amount of an agent effective to produce the intended pharmacological result. For example, a therapeutically effective amount is an amount sufficient to effect a beneficial therapeutic response in the patient over time (i.e., to treat a disease or condition or ameliorate the symptoms of the disease being treated in the patient). The amount actually administered will be dependent upon the individual to which treatment is to be applied, and will preferably be an optimized amount such that the desired effect is achieved without significant side effects. As described further in detail below, the dose may also be determined by the efficacy of the particular inhibitor or antagonistic agent employed and the condition of the patient, as well as the body weight or surface area of the patient to be treated. The size of the dose also will be determined by the existence, nature, and extent of any adverse side-effects that accompany the administration of, for example, a particular agent, vector or transduced cell type to a particular patient.

Therapeutically effective doses of agent(s) capable of preventing, inhibiting or reducing the incidence of telomerase/L1RT mediated cancer are readily determinable using data from cell culture assays disclosed herein and/or from in vivo assays using an animal model. The animal model can also be used to estimate appropriate dosage ranges and routes of administration in humans. Experimental animals bearing solid tumors of human origin (or art-accepted animal models) are frequently used to optimize appropriate therapeutic doses prior to translating to a clinical environment. Such models are known to be very reliable in predicting effective anti-cancer strategies. For example, mice bearing solid tumors or art-accepted mouse models are widely used in pre-clinical testing to determine working ranges of therapeutic agents that give beneficial anti-tumor effects with minimal toxicity. Due to the safety already demonstrated in art-accepted models, at least with respect to nucleoside analogs exemplified herein, pre-clinical testing of the present invention will be more of a matter of routine experimentation. In vivo efficacy may be predicted using assays that measure inhibition of tumor formation (progression), tumor regression or metastasis, and the like.

Exemplary in vivo assays of anti-tumor efficacy of compounds of the formulas (I) to (VI), ACV, PCV and/or GCV using nude mice subcutaneous (s.c.) tumors grown from the human HeLa cancer cell line (i.e., xenografts bearing mice) as cancer models are described below.

Human cancerous cells needed for in vivo assays may be prepared, for example, as follows: Telomerase positive HeLa human cell line and telomerase negative U-2 OS human cell line are obtained from public sources. Cells are maintained in D-MEM media supplemented with 10% foetal calf serum at 37° C. in a humidified atmosphere of 5% CO₂.

For in vivo assay, appropriate host, e.g., nude (nu/nu) mice of about 5-7 weeks old are obtained and maintained in pathogen-free conditions. Approximately, 1×10⁶ HeLa cells (and/or U-2 OS cells) contained in 200 μl of serum-free media are delivered to all animals, briefly anaesthetize with Metofane, by subcutaneous (s.c.) injection in flank. Then the mice are divided into experimental group and control group. Appropriate concentrations of compounds of the formulas (I) to (VI), ACV, PCV and/or GCV are used for tumor growth progression or regression assays.

In one embodiment, impairment of s.c. tumor growth or time to progression rather than decrease in size of an established tumor (regression) is assessed. In this embodiment, starting from the day zero, mice in the experimental group receive GCV in drinking water ad libitum. Concentration of GCV in water can be 2 mg/ml. Fresh solution of GCV is supplied every 3 days. Mice in the control group receive only drinking water. Tumors are measured every 2-3 days. Mice are sacrificed when tumors exceed 1 cm³. Tumor volume is calculated with formula 4/3πr³, where r is the radius of the tumor. All mice in the control group should develop tumors and all mice in the experimental group remain tumor free.

In another embodiment, the reagents and methods of the invention can be used to promote tumor regression in vivo in immunocompetent animal carrying pre-established tumors; i.e., the reagents of the invention can be used to treat animals with preexisting tumors. In this case, 10⁶ mouse hapatoma MH-22 cells or the like are injected subcutaneously in the flank of the C3HA mice to establish tumors. Once tumors are established after tumor cell implantation, the mice in the experimental group are administered with a composition containing Famvir i.g. solution in drinking water ad libitum, and the mice in the control group receive the same composition but without the drug (e.g., distilled water). Tumor growth is monitored every 2-3 days. When any of compounds of the formulas (I) to (VI) is administered for about 30 days to these tumor bearing animals, retarded tumor growth can be observed. Such inhibition of tumor cell growth is not observed in the control group. Few weeks after the start of the treatment, only the animals treated with compositions containing at least one of compounds of the formulas (I) to (VI), should show complete tumor regressions in a significant number of tumor bearing animals.

In another embodiment, in vivo assays that qualify the promotion of apoptosis may also be used. In this embodiment, xenograft bearing animals treated with the therapeutic composition may be examined for the presence of apoptotic foci and compared to untreated control xenograft-bearing animals. The extent to which apoptotic foci are found in the tumors of the treated animals provides an indication of the therapeutic efficacy of the composition.

In designing appropriate doses of agent(s) for the treatment of human telomerase-mediated cancers (both early stage tumors and vascularized tumors), one may readily extrapolate from the animal studies described herein in order to arrive at appropriate doses for clinical administration. To achieve this conversion, one would account for the mass of the agents administered per unit mass of the experimental animal and, preferably, account for the differences in the body surface area between the experimental animal and the human patient. All such calculations are well known and routine to those of ordinary skill in the art. Thus, the determination of a therapeutically effective dose is well within the capability of those skilled in the art.

For example, in taking the successful doses of compounds of the formulas (I) to (VI) in cell culture assays and in the mouse studies, and applying standard calculations based upon mass and surface area, effective doses for use in adult human patients would be between about 1000 mg and about 6000 mgs of a compound of the formulas (I) to (VI), per patient per day, and preferably, between about 500 mgs and about 1000 mgs per patient per day. Accordingly, using this information, it is contemplated herein that low doses of therapeutic agents (e.g., SN 1, SN 2, acyclovir, ganciclovir, penciclovir and the corresponding pro-drugs, i.e., valacicalovir, valganciclovir and famciclovir) for human administration may be about 1, 5, 10, 20, 25 or about 30 mgs or so per patient per day; and useful high doses of therapeutic agent for human administration may be about 250, 300, 400, 450, 500 or about 600 mgs or so per patient per day. Useful intermediate doses may be in the range from about 40 to about 200 mgs or so per patient.

Notwithstanding these stated ranges, it will be understood that, given the parameters and detailed guidance presented herein, further variations in the active or optimal ranges will be encompassed within the present invention. The intention of the therapeutic regimens of the present invention is generally to produce significant anti-tumor effects whilst still keeping the dose below the levels associated with unacceptable toxicity. In addition to varying the dose itself, the administration regimen can also be adapted to optimize the treatment strategy. A currently preferred treatment strategy is to administer between about 1-500 mgs, and preferably, between about 10-100 mgs of the inhibitor or antagonist of telomerase or therapeutic cocktail containing such, about −4 times within about a 60 days period. For example, doses would be given on about day 1, day 3 or 4 and day 6 or 7. Administration can be accomplished via single or divided doses taken orally or, for example, by administration to the site of a solid tumor directly or in a slow release formulation. The physician responsible for administration will, in light of the present disclosure, be able to determine the appropriate dose for the individual subject, the form and route of administration. Such optimization and adjustment are routinely carried out in the art and by no means reflect an undue amount of experimentation. In administering the particular doses themselves, one would preferably provide a pharmaceutically acceptable composition according to regulatory standards of sterility, pyrogenicity, purity and general safety to the human patient systemically. Physical examination, tumor measurements, and laboratory tests should, of course, be performed before treatment and at intervals up to one to few months after the treatment and one skilled in the art would know how to conduct such routine procedures. Clinical responses may be defined by any acceptable measure. For example, a complete response may be defined by the disappearance of all measurable tumors within a given period after treatment.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the patient undergoing therapy. It will ultimately be at the discretion of the attendant physician or veterinarian.

### WORKING EXAMPLES

The following working examples are provided to demonstrate preferred embodiments of the invention, but of course, should not be construed as in any way limiting the scope of the present invention. The examples below were carried out using conventional techniques that are well known and routine to those of skill in the art, except where otherwise described in detail Further, it should be appreciated by those of skill in the art that the techniques disclosed in the examples represent techniques found by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Direct Inhibition of Telomerase In Vitro by Triphosphates of Acyclic Nucleosides Analogs

Biosynthesis and isolation of acyclic analogs triphosphates as described (Agbaria R et al. Biosynthetic ganciclovir triphosphate: its isolation and characterization from ganciclovir-treated herpes simplex thymidine kinase-transduced murine cells. Biochem Biophys Res Commun. 2001, 289:525-30).

Briefly, 10⁷ U-2 OS cells were incubated for 48 h with 90 mM of ACV, or 45 mM of GCV, or 45 mM of PCV. Cells were washed with PBS and harvested by trypsinisation. After centrifugation the cell pellets were extracted with 60% methanol. The extracts were heated at 95° C. for 2 min following the evaporation under vacuum. Dry pellets were dissolved in 100 μl of PCR grade water.

Real time TRAP assay using HeLa cells extract was performed as described above. To demonstrate direct inhibition of telomerase by triphosphates of acyclic nucleoside analogs master mix for TRAP assay was supplemented with 5 μl of crude extracts from acyclic nucleoside treated U-2 OS cells.

Thus, it has been demonstrated that PCV-TP, GCV-TP and ACV-TP directly inhibit telomerase from 10 to 100 times under these conditions.

### Example 2

### Induction of Telomere Shortening, G2 Arrest and Apoptosis in Telomerase Negative Alt Cells and Telomerase Positive Cells

(a) Induction of telomere shortening, G2 arrest and apoptosis in telomerase negative ALT cells after AZT treatment or ganciclovir treatment were carried out as follows:

To detect L1 specific RNA in two cell lines (U-2 OS and Saos-2 osteosarcomas), reported to maintain telomeres by ALT mechanism, total mRNA was analyzed by dot blotting with an L1 retrotransposon specific probe. The reported telomerase-positive cell lines (HEC-1 and HeLa) were used for comparison. Both ALT cell lines (U-2 OS and Saos-2 osteosarcomas) were positive in this test. HEC-1 cells were completely negative, with only traces of L1 transcripts in HeLa cells, as previously reported.

The ALT cell lines were treated with therapeutic concentrations of AZT, to determine if slippage telomeric DNA synthesis could be inhibited by AZT-TP, and thereby induce telomere shortening. Telomere length in AZT treated and untreated cell lines was measured by flow cytometry with a telomere specific peptide nucleic acid (PNA) probe. To determine cell cycle distribution, cells were stained with propidium iodide (PI). After 14 days of AZT treatment, both ALT cell lines demonstrated telomere shortening, massive apoptosis and G2 arrest. To confirm the specificity of AZT-induced telomere shortening for ALT cells, a HeLa cell line, known to be positive for telomerase, was treated with AZT under the same conditions. AZT at the chosen concentration had no effect on telomere length or cell cycle distribution in the Hela cells.

To demonstrate telomere shortening and changes in DNA synthesis rate, dynamic, U-2 OS cells were treated with AZT for different amounts of time, and analyzed by flow cytometry simultaneously. Rate of DNA synthesis was determined by incorporation of 5-bromodeoxyuridine (BdU). Results showed progressive telomere shortening and decrease in DNA synthesis. It is important to note that changes in cell cycle distribution, DNA synthesis and telomere length were rapid and could be detected after only 10 days of AZT treatment.

At the same time, PI staining demonstrated a higher DNA content in AZT treated cells at later stages of treatment, compared to untreated cells. A rational explanation of this fact is a short telomere induced chromosome end-to-end joining. Induction of apoptosis in AZT treated ALT cells seems to be p53 independent since U-2 OS and Saos-2 represent both p53+/+ and p53−/− cancer cell lines.

Separately, U-2 OS cells were also treated with therapeutic concentrations of a guanine analog, ganciclovir (GCV), to demonstrate that the slippage telomeric DNA synthesis can be inhibited by GCV-TP and telomere shortening can be induced. Telomere length in untreated and GCV treated cells was measured by flow cytometry with a telomere-specific PNA probe as described above.

To determine cell cycle distribution, cells were stained with propidium iodide (PI). After 14 days of treatment with GCV at a concentration of 0.3 μg/ml, the U-2 OS cells demonstrated telomere shortening, massive apoptosis programmed cell death) and G2 arrest.

(b) Induction of telomere shortening, G2 arrest and apoptosis in telomerase positive cancer cells after ganciclovir (GCV) and acyclovir (ACV) treatments has been carried out as described below.

To detect telomerase specific activity in two cell lines (Hela and NuTu-19) real time TRAP assay was performed. The reported telomerase-positive cell lines (HeLa) was used for comparison. Both cell lines were positive in this test.

The telomerase positive cell lines were treated with therapeutic concentrations of GCV (1.5 μM or ACV (3.0 μM), to demonstrate that telomeric DNA synthesis could be inhibited within the cells, and thereby induce telomere shortening. Telomere length in GCV and ACV treated and untreated cell lines was measured by flow cytometry with a telomere-specific peptide nucleic acid (PNA) probe. To determine cell cycle distribution, cells were stained with propidium iodide (PI). After 14 days of both kinds of treatment, both cell lines demonstrated telomere shortening, massive apoptosis and G2 arrest.

To demonstrate changes in cell cycle distribution HeLa and NuTu-19 cells were treated with GCV or ACV for 14 days stained with PI, and analyzed by flow cytometry simultaneously. Results showed G2 arrest of cell cycle. It is important to note that changes were rapid and could be detected after only 14 days of ACV treatment. In contrast, the nucleoside analog, AZT had no effect on telomere length or cell cycle distribution in telomerase positive cells, HeLa and NuTu-19, even at elevated concentrations e.g., 100 μM.

At the same time, PI staining demonstrated a higher DNA content in GCV or ACV treated cells at later stages of treatment, compared to untreated cells. A rational explanation of this fact is a short telomere induced chromosome end-to-end joining.

The origin of the cell lines are uterine cervix (HeLa) and epithelial ovarian (NuTu-19). Cells were cultured in D-MEM media supplemented with 10% fetal calf serum at 37° C. in a humidified atmosphere of 5% CO₂. For treatment of the cells with GCV, the media was supplemented with 1.5 μM of GCV (Cymevene, Hoffman-La Roche). For treatment of the cells with ACV, the media was supplemented with 3 μM of Acyclovir (Acyclovir, TEVA Pharm. Ind. Id. Israel).

Real time TRAP assay was performed as described (Wege et al., SYBR Green real-time telomeric repeat amplification protocol for the rapid quantification of telomerase activity. Nucleic Acids Res. 2003; 31(2):E3-3). For telomere length measurement by flow cytometry, cells were stained with telomere specific FITC conjugated (C₃TA₂)₃ PNA (Applied Biosystems) probe and contrastained with 0.06 μg/ml PI as described by Rufer, N., Dragowska, W., Thornbury G., Roosnek, E., Lansdorp P. M. Telomere length dynamics in human lymphocyte subpopulations were measured by flow cytometry. Nat. Biotechnol. 16, 743-747 (1998)).

Thus, it has been demonstrated herein that the nucleoside analogs GCV and ACV clearly block telomerase positive cancer in widely accepted model systems.

(c) Induction of telomere shortening, G2 arrest and apoptosis in telomerase positive cancer cells after acyclovir (ACV) ganciclovir (GCV) and penciclovir REV) and treatments have been carried out as described below.

Both telomerase positive (Hela) telomerase negative (U-2 OS) cell lines were used. Appropriate assays were performed to detect and confirm telomerase/L1RT specific activity in these cells. The cell lines were treated with therapeutic concentrations of ACV (3.0 E, GCV (1.5 μM) or PCV (1.5 μM) to demonstrate that telomeric DNA synthesis could be inhibited within the cells, and thereby induce telomere shortening. Telomere length in ACV, GCV and PCV treated and untreated cell lines was measured by flow cytometry with a telomere—specific peptide nucleic acid (PNA) probe. To determine cell cycle distribution, cells were stained with propidium iodide (PI). After 10 and 14 days of treatments, both cell lines demonstrated telomere shortening, massive apoptosis and G2 arrest.

To demonstrate changes in cell cycle distribution, HeLa and U-2 OS cells were treated with ACV, GCV or PCV for 14 days stained with PI, and analyzed by flow cytometry simultaneously. Results showed G2 arrest of cell cycle. It is important to note that changes were rapid and could be detected after only few days of ACV treatment.

The U-2 OS (osteosarcoma) and HeLa (uterine cervix) cell lines used in this study were obtained from American Type Culture Collection (Rockville, Md.). Cells were cultured in D-MEM media supplemented with 10% fetal calf serum at 37° C. in a humidified atmosphere of 5% CO₂. For treatment of the cells with ACV, the media was supplemented with 3 μM of acyclovir (acyclovir, TEVA Pharm. Ind. Ltd, Israel). For treatment of the cells with GCV, the media was supplemented with 1.5 μM of GCV (Cymevene, Hoffman-La Roche). For treatment of the cells with PCV, the media was supplemented with 1.5 μM of PCV (penciclovir, Merck & Co.).

Real time TRAP assay was performed as described (Wege et al., SYBR Green real-time telomeric repeat amplification protocol for the rapid quantification of telomerase activity. Nucleic Acids Res. 2003; 31(2):E3-3). For telomere length measurement by flow cytometry, cells were stained with telomere specific FITC conjugated (C₃TA₂)₃ PNA (Applied Biosystems) probe and contrastained with 0.06 μg/ml PI as described by Rufer, N., Dragowska, W., Thornbury G., Roosnek, E., Lansdorp P. M. Telomere length dynamics in human lymphocyte subpopulations were measured by flow cytometry. Nat. Biotechnol. 16, 743-747 (1998)).

Thus, it has been demonstrated herein that the nucleoside analogs ACV GCV and PCV clearly cause decrease in telomere lengths.

### Example 3

### Prevention of Telomerase Negative (ALT) Tumor Development

(a) Nude mice injected with U-2 OS/RAS cells: Crl1:CD1-/nu mice were purchased from Charles River Laboratories, Charles River Deutschland GmbH (Dec. 23, 2004). A total of 12 Nude mice were injected s.c. with 6×10⁵ U-2 OS/RAS cells. These mice were divided into experimental and control groups.

The experimental group of mice started to receive AZT in drinking water (1 mg/ml) from the day one. In the control group, mice developed tumors (in N1 by the 26^{th} day; in N2 by the 340 day and in N3 by the 41^{st} day following the tumor cell injection).

All mice without tumors were sacrificed. One mouse from control group that developed tumors first was sacrificed on the 51 day following the tumor cell injection. Tumor tissue was mechanically separated to raise cell suspension. About 20 million of cells were seeded in McCoy's 5 A media supplemented with 10% FCS. The tissue cultured cells were used for further analysis.

Two mice from control group with tumors started to receive AZT in drinking water (1 mg/ml) from day 52. One mouse had died on day 80. Second was sacrificed on day 110 following the tumor cell injection. Tumor tissue was collected and stored at −80° C. for further analysis.

1. Three mice out of six in control group had developed ALT tumors. No one mice from AZT treated group had developed tumors.

2. Tissue culture that was developed from ALT tumor is telomerase positive. It indicates that inside telomerase negative tumor, some cells spontaneously activate telomerase.

3. Mice with ALT tumors treated with AZT demonstrated slowing of tumor growth.

(b) Nude mice injected with HeLa cells: Nude mice were injected s.c. with HeLa cells (3×10⁵) to demonstrate prevention of development and treatment of telomerase positive tumors in vivo. Experimental group received valganciclovir in drinking water from day 0. Human cancer HeLa cell culture was purchased from ATCC. In all, 12 CD1/-nu and 12 NMRiI/-nu nude mice were purchased from Charles River Laboratories, Charles River Deutschland GmbH. These nude mice were injected s.c. with 3×10⁵ Hela cells. Mice in the experimental groups (6 mice per strain) were exposed to Valcyte® (val-ganciclovir) in drinking water (1 mg/ml) from day 0.

All mice in control and treated groups had developed tumors. In about 14 days, all mice were bearing the tumors. The tumor in one mouse from the treated group began to regress and, by about the 30& day, this tumor was eliminated by monotherapy with Valcyte®. Other mice in the treated groups demonstrated slowing of tumor growth.

### Example 4

### Switching of ALT Mechanism to Telomerase-Dependent Mechanism of Telomere Maintenance

The effects of AZT herein described on growth, cell cycle and telomeres were examined in U2-OS cells and HeLa cells. U2-OS cells, which are telomerase negative, express L1RT and Hela cells, which are telomerase positive, express telomerase.

(a) In vitro arrays: U-2 OS cells were incubated with 0.2 μM of AZT for 22 days. Actively growing clones were analyzed by flow-FISH with telomere specific PNA probe, and in real time TRAP assay using HeLa cells as positive control. Isolated actively growing clones were different from parental cells in DNA content and telomere length. Also all isolated actively growing clones were positive for telomerase in TRAP assay.

(b) In vivo assays: The nude mouse from control untreated group that developed tumor from modified human U-2 OS osteosarcoma was sacrificed. Tumor tissue was mechanically separated to cell suspension. 20 millions of cells were seeded in McCoy's 5 A media supplemented with 10% FCS. Few cells had attached to the plastic surface giving the rise of tissue culture that analysed in real time TRAP assay using HeLa cells as positive control. The human origin of developed tissue culture was proved by RT-PCR. The use of selective primers demonstrated the presence of human and the absence of mouse GAPDH mRNA.

The results clearly show that tissue culture that was developed for from ALT tumor is telomerase positive. It indicates that inside telomerase negative tumor some cells spontaneously activate telomerase.

In other words, treatment of telomerase negative cancer cells with AZT allows selection of positive cells and cancer can relapse.

### Example 5

### Demonstration of Tumor Size Reduction in Mice Following the Administration of Double Cocktail, Valganciclovir and Zidovudine (AZT) or Valacyclovir and Retmvir®

This example illustrates the anti-tumor activity and efficacy of double cocktails in mouse models.

(a) In vivo assays using Valganciclovir and zidovudine (AZT) combination: Human cancer HeLa cell culture was purchased from ATCC. A total of 24 CD1/-nu nude mice were purchased from Charles River laboratories, Charles River Deutschland GmbH. These mice were injected s.c. with 1×10⁵ HeLa cells. The tumor volumes were determined by measurement of maximal and minimal diameters of the tumor. The calculations were conducted using the formula V=4/3 πRₘₐₓ×Rₘᵢₙ², where Rₘₐₓ−½ of maximal diameter of the tumor, and Rₘᵢₙ−½ of minimal diameter of the tumor. nude mice were injected s.c. with 1×10⁵ Hela cells.

All mice had developed tumors by 35^{th} day after the injection at which time 12 mice from treated group were administered with double cocktail of Valcyte® and Retrovir® at concentration 1 mg/ml each in drinking water. Of the 12 treated mice, after 20 days of double cocktail treatment, six mice were administered with a triple cocktail (Valcyte®+Retrovir®+ddI at the dose of 1 mg/ml each in drinking water for a further period 21 days (see Example 7 below) and only six mice continued to receive the double cocktail for another 21 days at which time tumor measurements were taken for all mice injected with HeLa cells. Of the remaining 12 mice injected with HeLa cells but untreated with any cocktail or anticancer agent thus far, six were maintained as controls for the rest of the duration of the experiment. The other six were treated with Xeloda® 67 days after the injection of HeLa cells (see Example 7 below).

The mice from the double cocktail treated group showed 50% of survival. Tumor volumes in the treated and untreated mice are presented in the Table for this example. One mouse showed 993% tumor size reduction compared to untreated treated group. One mouse demonstrated 98.9% tumor size reduction compare to untreated treated group and one mouse demonstrated 95.8% tumor size reduction compare to untreated treated group.

TABLE FOR EXAMPLE 5
Tumor regression in nude mice following
double cocktail treatment

| | | Tumor Volume (mm³) in |
|---|---|---|
| | | nude mice 75 days |
| | | following the injection of |
| Treatment | No. of mice* | HeLa cells |
| Valganciclovir + | mouse 1 | 14.1 |
| Zidovudine for 20 days | mouse 2 | 23.6 |
| | mouse 3 | 91.7 |
| Untreated control | 4 | 2178.0 (mean value) |

*All mice treated with Valgancilovir (Valcyte ®) and Retrovir ® manifested symptoms of toxicities perhaps due to incompatibility of Valcyte ® and Retrovir ® combination, which combination is known in the art to induce severe toxicities in some cases. Some or all of the side effects of the Valcyte ® and Retrovir ® combination could have been offset by using known protective agents and supportive therapy but these were not used on the mice in the instant experiment.

(b) In vivo assays using Valacyclovir and zidovudine (AZT) combination: Mouse hepatoma MH22A was purchased from the tissue culture collection of the Institute of Cytology (Russian Academy of Medical Science, Petersburg, Russia). The frozen cells were defrosted, transferred into the culture medium MEM supplemented with 10% foetal calf serum. The cells were grown at 37° C. under a humidified atmosphere of 5% CO₂. To determine the telomerase status of MH22A cells, these cells were analysed in real time TRAP assay as described above using Hela cells as positive control. The results clearly showed that MH22A cells are telomerase positive.

Eight weeks old male C3HA inbred mice were purchased from Laboratory Animals Breading Facility of Russian Academy of Medical Science (Rappalovo, Leningrad region). About 2×10⁵ MH22a cells were injected s.c. into back flank of 35 mice. After the injections, the mice were randomly divided into different experimental groups.

(a) A group of 7 mice started to receive valaciclovir (Valtrex®, GlaxoSmithKline) from day 0 at concentration 1 mg/ml in drinking water. On day 30, the mice started to receive the mixture of 1 mg/ml of valaciclovir, 1 mg/ml of AZT and 1 mg/ml of Xeloda® in drinking water.

(b) A group of 7 mice started to receive the mixture of 1 mg/ml of valaciclovir and 1 mg/ml of AZT (Retrovir® LV., GlaxoSmithKline) from day 0 in drinking water.

(c) From day 14, a group of 7 previously untreated mice started to receive the mixture of 1 mg/ml of valaciclovir and 1 mg/ml of AZT in drinking water.

(d) From day 16, one group of 7 previously untreated mice started to receive the mixture of 1 mg/ml of valaciclovir and 1 mg/ml of AZT in drinking water. From day 30 onwards, mice started to receive the mixture of 1 mg/ml of valaciclovir, 1 mg/ml of AZT and 1 mg/ml of Xeloda® in drinking water.

(e) A group of 7 untreated mice was serving as positive control.

All mice form the control groups had developed tumors 5-8 nm in diameter by day 14. On day 17 of the experiment, 5 mice from the group that had received the combination of Valcyte® and AZT were tumor free. Two mice from the same group had tumors 3 mm in diameter. All mice from the other group had developed the tumors with average size 10 mm in diameter.

By day 45 of the experiment, seven mice receiving valaciclovir alone from day 0 and seven mice receiving the combination of Valtrex® and Retrovir® from day 16 started to receive Xeloda® in drinking water at a concentration of 1 mg/ml.

By day 60 of the experiment, four out of seven mice receiving the combination of Valtrex® and Retrovir® from day 0 remained tumor-free. In all other treated groups, only two mice remained tumor-free. All control mice were bearing the tumors much bigger in size that those in the treated groups.

By day 75 of the experiment, the mice in the control and treatment groups started to die. By day 85 of the experiment, all mice in the control untreated group died. Only four mice in the prevention group (Valtrex® and Retrovir® from day 0) and two mice in each treatment groups (Valtrex® from day 0 and Xeloda® from day 45, Valtrex® and Retrovir® from day 16 and Xeloda® from day 45, Valtrex® and Retrovir® from day 14) were alive and tumor free. At this point in time, all treatments were discontinued. By day 150 of the experiment, the 4 mice in the prevention group and 2 mice in each of the treatment groups were still alive and tumor-free.

### Example 6

### Demonstration of Telomere Shortening in Cultured Cells Grown in a Medium Exposed to the Triple Cocktail, Acyclovir+AZT+ddI

Treatment of telomerase negative ALT cells (U-2 OS) with AZT and ACV for 28 days allowed selection for proliferating cells that are resistant to both drugs. The treatment of those selected cells with the combination of AZT, ACV and 0.01 mg/L of ddI induced progressive telomere shortening and apoptosis.

### Example 7

### Demonstration of Tumor Size Reduction in Mice Following the Administration of Double Cocktail, Valcyte® and Retrovir® or Valtrex® and Retrovir®

This example illustrates the potential of triple cocktail to enhance the efficacy of DNA-damaging chemotherapeutic agent by selectively increasing the sensitivity of tumor cells in mouse models.

As mentioned in Example 5 above, a total of six mice, after 20 days of double cocktail treatment, were administered with a triple cocktail (Valcyte®+Retrovir®+ddI at the dose of 1 mg/ml each in drinking water) for a further period 21 days. After 14 days of triple cocktail treatment, Xeloda® at a concentration 1 mg/ml was added to the triple cocktail. Six mice previously untreated with any drug for 67 days after the injection of HeLa cells were treated with Xeloda® at a concentration 1 mg/ml in drinking water. Tumor measurements were taken in all mice 75 days following the injection of HeLa cells.

The mice from control untreated and only Xeloda® treated groups showed 66% survival. The average size of tumors in Xeloda® only treated group of mice was 17% less compared to the untreated control group.

Mice treated with a combination of the triple cocktail and Xeloda® showed 100% survival rate. Tumor volumes in the treated and untreated mice are presented in the Table for this example. In two of the six mice in the group, tumors vanished completely. In the remaining four mice, tumors could be determined only by the palpation. In terms of tumor reduction, two mice showed 99.8% tumor reduction compared to Xeloda® only treated group. One mouse demonstrated 98.2% tumor size reduction compared to Xeloda® only treated group. One mouse demonstrated 90.0% tumor size reduction compared to Xeloda® only treated group.

TABLE FOR EXAMPLE 7
Tumor regression/elimination in nude mice following treatment
with a combination of cocktails and a genotoxic agent

| | | Tumor Volume (mm³) in |
|---|---|---|
| | | nude mice 75 days |
| | | following the injection of |
| Treatment | No. of mice* | HeLa cells |
| Valcyte ® + Retrovir ® for | mouse 1 | 0.0 |
| 20 days followed by | mouse 2 | 0.0 |
| Valcyte ® + Retrovir ® + | mouse 3 | 4.2 |
| ddI | mouse 4 | 4.2 |
| for 14 days followed by | mouse 5 | 33.5 |
| Valcyte ® + Retrovir ® + | mouse 6 | 179.8 |
| ddI + Xeloda ® for 8 days | | |
| Xeloda ® for 8 days | 4 | 1811.0 (mean value) |
| Untreated control | 4 | 2178.0 (mean value) |

*All mice treated with Valcyte ® and Retrovir ® manifested symptoms of toxicities perhaps due to incompatibility of Valcyte ® and Retrovir ® combination, which combination is known in the art to induce severe toxicities in some cases. Some or all of the side effects of the Valcyte ® and Retrovir ® combination could have been offset by using known protective agents and supportive therapy but these were not used on the mice in the instant experiment.

### Example 8

### Treatment of a Human Patient Using Cytotoxic Tumor Therapy (Background Therapy) with Genotoxic Chemotherapy Intervention

This example illustrates therapeutic efficacy of background therapy (using a double or triple cocktail) interspersed with DNA-damaging genotoxic chemotherapy in a human patient. The patient is female, age 65, diagnosed with inoperable carcinoma of stomach as more fully described below.

The patient, after complaints of stomach pain, weight loss, nausea, and vomiting, was diagnosed with diffuse infiltrated stomach cancer, grade IV with Ascitis. As part of the diagnosis, a combination of clinical, radiological, and surgical procedures were carried out. These evaluations helped in defining the cancer stage of this patient and provided an insight into prognosis and a sound basis for planning the therapy.

More specifically, as part of the initial examination, Fibrogastroscopy was performed on this patient. During this initial examination, it was found that the stomach of the patient was deformed, rigid and was relatively indistensible on air insufflation. Also, in the upper two thirds of the stomach, bleeding tissue was detected. The antrum was without any pathology. Multiple biopsies were taken from the stomach tissue. From the histological analysis, the patient was found to have adenocarcinoma of stomach with low grade of differentiation. The X-ray examination of the stomach with the use of contrast revealed the following clinical features: starting from the subcardial part of the stomach to the lower third of the stomach, the walls were rigid. The diameter of the stomach in the middle third was 2.5 centimetres. The length of the tumor on the minor curvature was 7 centimetres and on the major curvature ranged from 11-12 centimetres. In the middle of major curvature of the stomach, an ulcer was also detected. The antrum and duodenum were without any pathology. A diagnostic laparoscopy was also performed. It revealed a total canceromatosis of parietal and visceral peritoneum. The cancer was found to be inoperable.

The patient was pretreated with the background therapy for 30 days followed by 53 days of background therapy interspersed with Xeloda® treatment as described further below:

(a) Background therapy: 1 tablet of Retrovir® (AZT) 300 mg and 1 tablet of Zovirax™ (acyclovir, Glaxo Welcome), 400 mg/Valcyte® (valganciclovir) 450 mg/Valtrex® (Val-ACV) 500 mg all BID. Different acyclic nucleoside analogs were used during the 83 days of background therapy for reasons related to market availability. The background therapy was initiated with acyclovir, which was used for 9 days, followed by Valcyte® for 30 days and then continued with Valtrex® for the remaining period of the treatment. These drugs were administered orally 2 hours before and 2 hours after the food intake. The background therapy was continued for 11.9 weeks.

(b) DNA damaging therapy: From day 31 of the background therapy, Xeloda® was administered orally at a dose of 4 tablets 500 mg (2 g) BID (i.e., 4 g a day—total) together with the background therapy drug. The intervention with Xeloda® consisted of three courses of Xeloda® with first course for 9 days followed by 14 days of break, then the second course for 7 days followed by 14 days of break, and then the final course for 10 days. Throughout this period the background therapy was maintained.

In about 4 weeks following the cessation of the above cancer treatment, laparotomy was performed. About 2 litres of ascetic fluid was found in the abdominal cavity. However, no signs of peritoneal canceromatosis were detected. The liver and diaphragm were without pathologic changes. The walls of the stomach were rigid during palpation. However, stomach serosa was without pathologic signs.

The patient was monitored periodically for over one year by routine clinical and imaging examinations. These examinations confirmed that the positive dynamic of the disease was stabilized or kept under control.

The genotoxic chemotherapy using Xeloda® caused toxicity in the patient and, therefore, could not have been given indefinitely until the patient achieved complete response. In contrast, the amounts of double cocktail and triple cocktail components maintained in the patient as a background therapy for the maintenance time period were either minimally or not toxic to the patient. It was possible to administer a given cocktail of drugs over a long period of time and combat cancer without the fear of tumor re-growth during the periods of suspension of Xeloda® treatment. It is thus clear that the background therapy, with its ability to affect telomere maintenance or induce telomere shortening, G₂/M arrest and/or massive apoptosis in tumor cells, provided for a favorable risk-to-benefit ratio, prevented tumor growth and survival advantage.

In short, the cancer would have been in its acute stages had the patient not been on the background therapy disclosed herein. The response to treatment by the patient, suffering from inoperable carcinoma of stomach, is truly astonishing since favorable responses to inoperable carcinoma of stomach are extremely uncommon.

While the human patient data is provided herein for the successful treatment of stomach carcinoma with a triple cocktail containing nucleoside analogs and using the disclosed treatment protocols, it is to be understood that the treatment protocol with routine modifications can be used for the treatment of other forms of cancer with a combination of inhibitors of the present invention, such as melanoma, NSCLC, renal cell carcinoma, and other cancers known in the art and exemplified herein. Further, the present invention provides an improvement in the treatment of all types of cancer, which can be treated with DNA-damaging cancer therapeutic agents including genotoxic chemotherapeutic agents, since by use of the administration protocol of the present invention, lower toxicities and/or less time is required than that associated with prior art protocols for administering antineoplastically effective amounts or doses of DNA-damaging agents.

### Example 9

### Preparation of the Acyclic Nudeoside Analogs and Prodrugs of the Invention

The acyclic nucleoside analogs of the present invention can be prepared following synthetic methodologies well-established in the practice of nucleoside and nucleotide chemistry. Reference is made to the following text for a description of synthetic methods used in the preparation of the compounds of the present invention: “Chemistry of Nucleosides and Nucleotides,” L. B. Townsend, ed., Vols. 1 3, Plenum Press, 1988, which is incorporated by reference herein in its entirety.

The acyclic nucleoside analogs of formulas I-IV of the present invention were prepared according to procedures detailed in the following examples. The examples are not intended to be limitations on the scope of the instant invention in any way, and they should not be so construed. Those skilled in the art of nucleoside and nucleotide synthesis will readily appreciate that known variations of the conditions and processes of the following preparative procedures can be used to prepare these and other compounds of the present invention. All temperatures are degrees Celsius unless otherwise noted.

(a) Synthesis of Formula (I) or SN 1

### 2-Hydroxyethylbenzoate

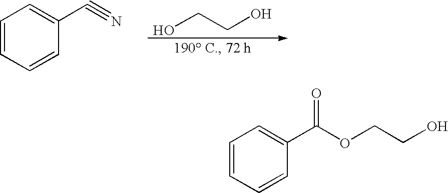

Benzonitrile (200 ml) and redistilled ethane-1,2-diol (600 ml) (protected from moisture; NaOH) were refluxed until evolution of ammonia ceased (3 days), and then cooled. Water (4000 ml) was added and the liberated oil extracted with ether (3×200 ml). After drying and removal of the solvent the residue was rectified through laboratory column at reduced pressure. This rectification gave 241 g (74%) of 2-hydroxyethylbenzoate, boiling range 160-162° C. (14 mm).

### 2-(Chloromethoxy)ethyl Benzoate

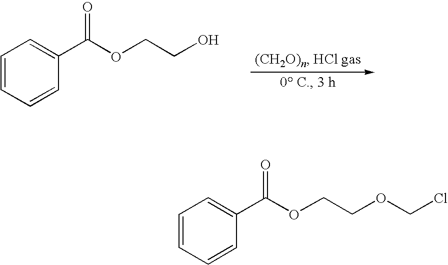

Dry HCl gas was passed through a mixture of 50 g (0.3 mol) of 2-hydroxyethylbenzoate in 200 ml of dry C₂H₄Cl₂ and 40 g (0.44 mol) of paraformaldehyde at 0° C. with stirring for 3 h. The solution was dried over CaCl₂ for 18 h, filtered, and evaporated in vacuo. The residual oil was distilled to give 525 g (93%) of 2-(chloromethoxy)ethyl benzoate, bp 126-129° C. (0.5 mm).

### 1-[[2-(Benzoyloxy)ethoxy]methyl]-5-methyluracil

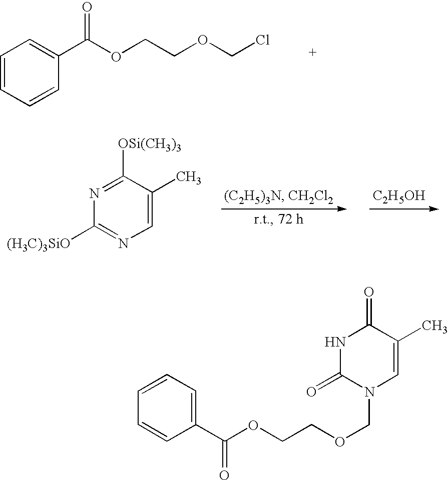

A mixture of 6.30 g (50 mmol) of thymine, 100 ml of hexamethyldisilazane, and 1 ml of trimethylsilylchloride was refluxed with stirring under nitrogen for 20 h. The resultant solution was spin evaporated in vacuo to an oil. To the residual oil in 50 ml of dichloromethane was added 10.60 g (49.4 mmol) of 2-(chloromethoxy)ethyl benzoate. The solution was cooled on ice and 11 ml (80 mmol) of triethylamine in 50 ml of dichloromethane was added rapidly dropwise. The resultant solution was stirred at ambient temperature for 72 h. The reaction was poured over 300 ml of aqueous (1:1) ethanole solution and shaken in a separatory funnel. The resultant mixture was diluted with 500 ml of water. The organic layer was separated, filtered and spin evaporated in vacuo. The residue crystallized under cyclohexane containing a little EtOAc: yield 9.29 g (61%); mp 94-99° C., which contained some impurities. Several recrystallizations from EtOAc gave pure material: yield 4.60 g (30%); mp 115-116° C. NMR (500 MDz, DMSO-d₆) δ, 1.77 (s, 3H) 3.87 (t, 2H), 4.39 (t, 2H) 5.13 (s, 2H), 7.44-7.98 (2 t+d, 5H), 11.16 (s, 1H).

### 1-[(2-Hydroxyethoxy)methyl]-5-methyluracil

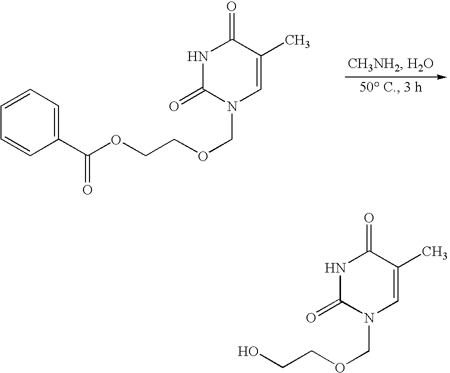

A solution of 0.870 g (2.86 mmol) of 1-[[2-(benzoyloxy)ethoxy]methyl]-5-methyluracil and 20 ml of 40% aqueous methylamine was heated on a bath at 50° C. for 3 h. The reaction was cooled and spin evaporated in vacuo. The residual syrup was triturated with Et₂O to give a solid, which was digested with Et₂O to remove N-methylbenzamide. The white solid was collected and washed with Et₂O: yield 0.495 g (86%); mp 138-140° C. Recrystallisation from EtOAc gave analytically pure material: yield 0.375 g (65%); mp 139-140° C.

NMR (500 MHz, DMSO-d₆) δ, 1.81 (s, 3H), 3.52 (s, 4H), 4.37 (br s, 1H), 5.07 (s, 2H), 7.41 (s, 1H), 11.06 (s, 1H).

(b) Synthesis of Formula (1) or SN 2

### 9-[(2-(Benzoyloxy)ethoxy)methyl]-adenine

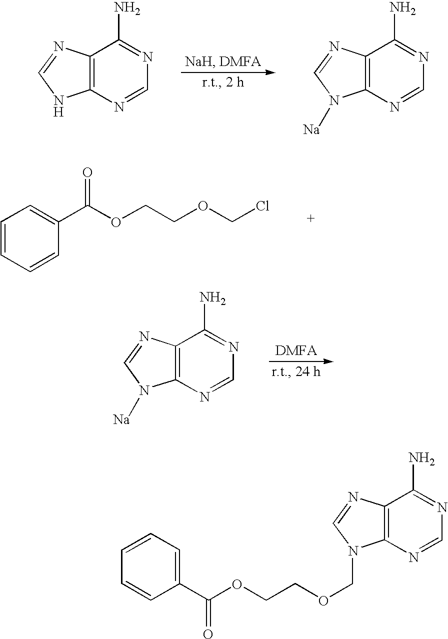

A 60% dispersion of sodium hydride in paraffin, (3.26 g, 82 mmol) was washed with hexane (3×10 ml) then suspended in DMF (250 ml) at 0° C. To this was added adenine (10.0 g, 74 mmol) slowly with stirring. On completion of addition the reaction mixture was warmed to room temperature and stirred for 2 h before 2-(chloromethoxy)ethyl benzoate (24 g, 15 eq.) was added over 3 n with continual stirring. The reaction mixture was stirred at room temperature for a further 24 h then the solution was concentrated to a paste. Water (100 ml) was added and the precipitate collected and recrystallised from 1-butanol, to give the title ester (8.1 g, 35%).

NMR (500 MH, DMSO-d₆) 6, 3.91 (t, 2H), 4.37 (t, 2H), 5.64 (s, 1H), 6.93 (br. s, 2H), 7.44-7.89 (2 t+d, 5H), 8.118 (s, 1H), 8.123 (s, 1H).

### 9-[(2-Hydroxyethoxy)methyl]-adenine

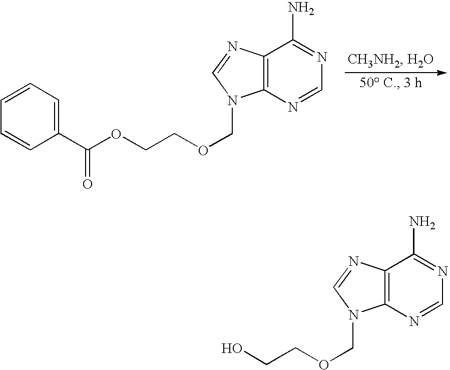

A solution of 1.2 g (3.83 mmol) of 9-[[2 (benzoyloxy)ethoxy]methyl]-adenine and 20 ml of 40% aqueous methylamine was heated on a bath at 50° C. for 3 h. The reaction was cooled and spin evaporated in vacuo. The residual syrup was triturated with Et₂O to give a solid, which was digested with Et₂O to remove N-methylbenzamide. The white solid was collected and washed with Et₂O: yield 0.672 g (84%).

Recrystallization from 1-butanol gave analytically pure material: yield 0.600 g (75%). NMR (500 M DMSO-d₆) δ, 3.51 (t, 2H), 3.55 (t, 2H), 4.44 (s, 1H), 5.59 (s, 2H), 6.89 (br. s, 2H), 8.12 (s, 2H).

### (c) Synthesis of Formula (III) or SN 3

### 1,3-Di-O-benzylglycerol

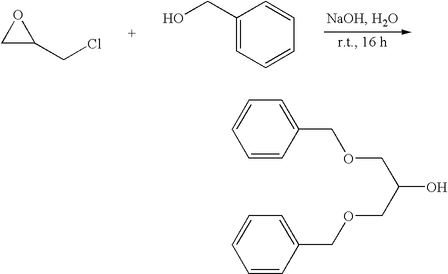

A solution of sodium hydroxide (100 g, 2.5 mol) in water (200 mL) was added over 10 min to benzyl alcohol (400 g, 3.9 mol). The mixture was cooled to 25° C., and then epichlorohydrin (100 g, 1.08 mol) was added with rapid stirring over 30 min. Vigorous stirring was continued for 16 h. The mixture was then diluted with water (1000 mL) and extracted with toluene (3×500 mL). The toluene extract was washed with water (500 mL), dried over Na₂SO₄ and evaporated to an oil, which was distilled to yield 150 g (50%) of 1,3-di-O-benzylglycerol, bp 155° C. (0.5 mm).

### 2-O-Chloromethyl,1,3-di-O-benzylglycerol

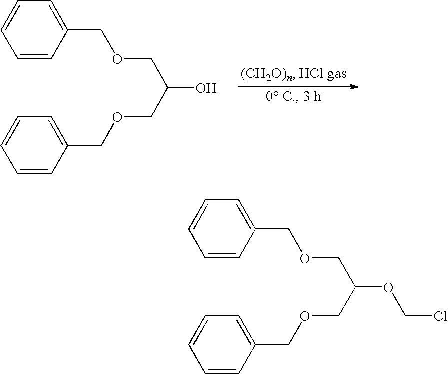

Hydrogen chloride gas (dried through concentrated H₂SO₄) was bubbled into a stirred mixture of paraformaldehyde (32 g, 0.8 mol) and 1,3-di-O-benzylglycerol (100 g, 037 mol) in methylene chloride (1000 mL) at 0° C. until all the solid dissolved (3 h). The resulting solution was stored at 0° C. for 16 h, dried over MgSO₄, and then evaporated to give 2-O-chloromethyl,1,3-di-benzylglycerol as a very unstable clear oil.

### 1-[(1,3-Dibenzyloxy-2-propoxy)methyl]-5-methyluracil

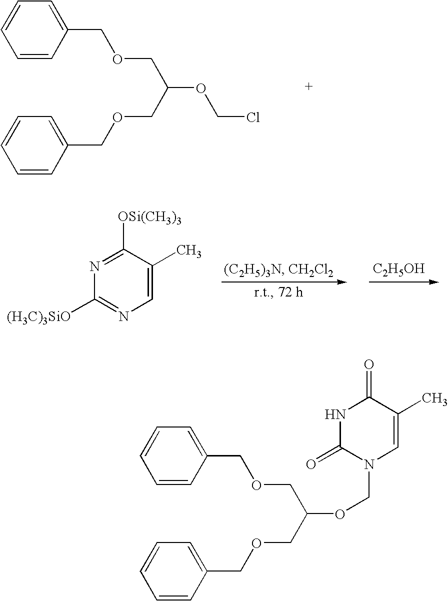

A mixture of 20 g (159 mmol) of thymine, 100 ml of hexamethyldisilazane, and 1 ml of trimethylsilylchloride was refluxed with stirring under nitrogen for 20 h. The resultant solution was spin evaporated in vacuo to an oil. To the residual oil in 300 ml of dichloromethane was added 56 g (175 mmol) of 20-chloromethyl,1,3-di-O-benzylglycerol. The solution was cooled on ice and 33 ml (240 mmol) of triethylamine in 60 ml of dichloromethane was added rapidly dropwise. The resultant solution was stirred at ambient temperature for 72 h. The reaction was poured over 500 ml of aqueous (1:1) ethanole solution and shaken in a separatory funnel. The resultant mixture was diluted with 500 ml of water. The organic layer was separated, filtered and spin evaporated in vacuo. The residue crystallized under hexane containing a little EtOAc. Several recrystallizations from EtOAc gave pure material: yield 17.6 g (27%); mp 96-98° C.

NMR (500 MHz, DMSO-d₆) δ 1.74 (s, 3H), 3.51 (m, 4H), 3.99 (m, 1H), 4.48 (s, 4H), 5.17 (s, 2H), 7.26 (s, 10H), 7.53 (s, 1H), 11.12 (s, 1H).

### 1-[(1,3-Dihydroxy-2-propoxy)methyl]-5-methyluracil

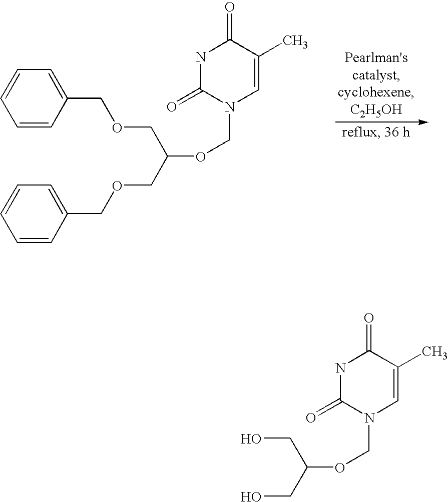

A mixture of 1-[(1,3-dibenzyloxy-2-propoxy)methyl]-5-methyluracil (30 g, 73 mmol), 20% palladium hydroxide on carbon (Pearlmans catalyst) (1000 mg), cyclohexene (400 mL), and ethanol (200 mL) was heated at reflux under N₂. After 8 and 24 h, additional catalyst (250 mg) was added. After 36 h, the solution was cooled to room temperature and filtered. The filtrate was evaporated, and the residue was triturated with benzene (100 mL) to give 14 g (83%) of crude 1-[(1,3-dihydroxy-2-propoxy)methyl]-5-methyluracil. Recrystallizations from 1-butanol gave pure material: mp 156-157° C.

NMR (500 MHz, DMSO-d₆) δ, 1.81 (s, 3H), 3.37 (m, 2H), 3.44 (m, 2H), 3.53 (m, 1H), 4.33 (br. s, 2H), 5.15 (s, 2H), 7.45 (s, 1H), 11.09 (s, 1H).

(d) Synthesis of Formula (IV) or SN 4

### 9-[(1,3-Dibenzyloxy-2-propoxy)methyl]-adenine

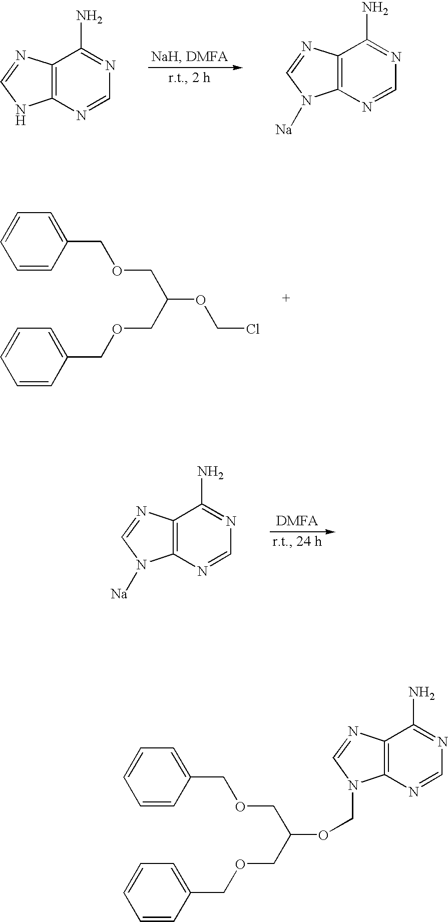

A 60% dispersion of sodium hydride in paraffin, (6.2 g, 156 mmol) was washed with hexane (3×100 ml) then suspended in DMF (500 ml) at 0° C. To this was added adenine (19 g, 141 mmol) slowly with stirring. On completion of addition the reaction mixture was warmed to room temperature and stirred for 2 h before 2-O-chloromethyl,1,3-di-benzylglycerol (68 g, 1.5 eq.) was added over 2 h with continual stirring. The reaction mixture was stirred at room temperature for a further 24 h then the solution was concentrated. Water (400 ml) was added and the precipitate collected and recrystallised from EtOAc. Several recrystallizations from EtOAc gave 17.2 g (29%) of the 9-[(1,3-dibenzyloxy-2-propoxy)methyl]-adenine.

NMR (500 MD, DMSO-d₆) δ, 3.45 (m, 2H), 3.50 (m, 2H), 4.10 (m, 1H), 4.42 (s, 4H), 5.68 (s, 2H), 6.93 (br. s, 2H), 7.21 (m, 5H), 7.27 (m, 5H), 8.08 (s, 1H), 8.13 (s, 1H).

### 9-[(1,3-Dihydroxy-2-propoxy)methyl]-adenine.

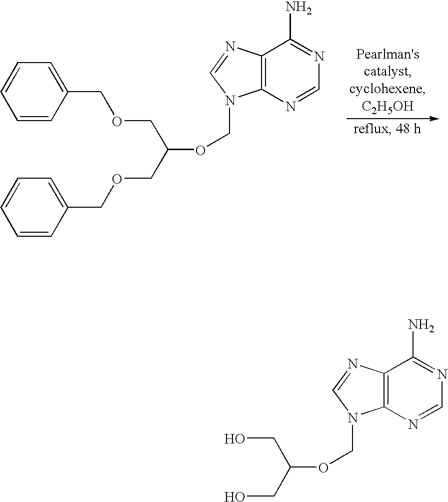

A mixture of 9-[(1,3-benzyloxy-2-propoxy) methyl]-adenine (35 g, 83 mmol), 20% palladium hydroxide on carbon (Pearlman's catalyst) (1000 mg), cyclohexene (400 mL), and ethanol (200 mL) was heated at reflux under N₂. After 16 and 36 h, additional catalyst (250 mg) was added. After 48 h, the solution was cooled to room temperature and filtered. The filtrate was evaporated and the residue was triturated with toluene (100 mL) to give 14 g (83%) of crude 9-[(1,3-dihydroxy-2-propoxy)methyl]-adenine. Recrystallizations from 1-butanol gave 13 g of pure material.

NMR (500 MHz, DMSO-d₆) δ, 3.36 (m, 2H), 3.45 (m, 2H), 3.61 (m, 1H), 4.43 (br. s, 2H), 5.69 (s, 2H), 6.98 (br. s, 1H), 8.15 (s, 1H), 8.18 (s, 1H).

(e) Synthesis of Formula (V) or SN5

### Triethyl 1,1,2-ethanetricarboxylate

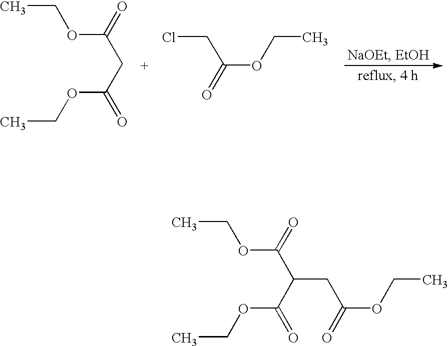

Sodium metal (23 g, 1 mol) was dissolved in dry ethanol (0.5 L) with stirring, then diethyl malonate (153 ml, 1 mol) was added for over 10 minutes. Ethyl chloroacetate (117 g, 0.95 mol) was then added dropwise to the stirred mature. On completion of the addition, the reaction mixture was heated under reflux for four hours then poured into 2 L of water and extracted with ether (3×500 mL). The ether fractions were combined, dried over MgSO₄, filtered and evaporated to give oil. This was vacuum distilled to give 197 g (84%) of triethyl 1,1,2-ethanetricarboxylate.

### 2-(Hydroxymethyl)butane-1,4-diol

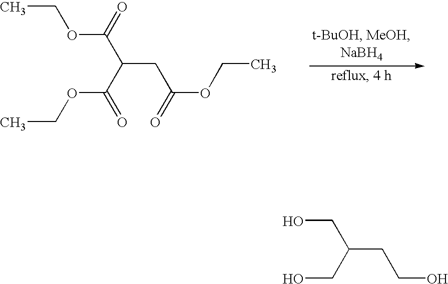

To a refluxing solution of 100 mL of triethyl 1,1,2-ethanetricarboxylate (108 g, 0.44 mol) and 50 g of sodium borohydride in 900 mL of dry tert-butanol, 100 mL of methanol was added dropwise over 150 minutes. The resulting solution was refluxed for a further 90 minutes and then was cooled to 10° C. 10% Hydrochloric acid was carefully added with vigorous stirring to neutralize the solution.

The solution was filtered and the inorganic residue was washed with 300 mL of dry ethanol. The organic solutions were combined and spin evaporated in vacuo.

The residue was extracted with 400 mL of absolute ethanol and the solution filtered. The solvent was removed under reduced pressure (0.5 mm) to afford 48 g (92%) of 2-(hydroxymethyl)butane-1,4-diol as a viscous clear oil.

### 5-(2-Hydroxyethyl)-2,2-dimethyl-1,3-dioxane

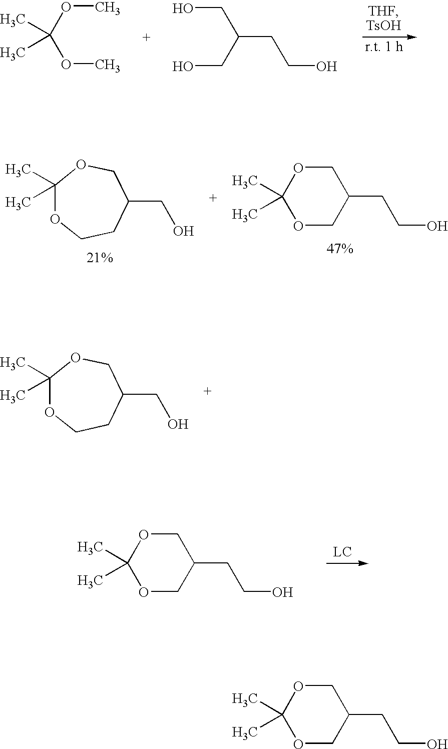

To a solution of 2-(hydroxymethyl)butane-1,4-diol (48 g, 0.4 mol) and 2,2-dimethoxypropane (55 g, 0.45 mol) in 200 mL of dry THF was added 3 g of p-toluenesulfonic acid monohydrate. The solution was stirred for 60 minutes at room temperature and was then neutralized by addition of triethylamine.

The solution was filtered and evaporated under reduced pressure. The residue was dissolved in 1000 ml of diethyl ether, filtered and reevaporated.

The resulting mixture consist of 47% 5-(2-hydroxyethyl)-2,2-dimethyl-1,3-dioxane, 21% seven-membered ring acetonide and other products (on GCMS data). The residue purified by column chromatography on silica gel eluting with 1-chlorobutane, chloroform and chloroform-methanol mixtures (25:1) to afford 24 g (38%) of 5-(2-hydroxyethyl)-2,2-diethyl-1,3-dioxane as a colorless liquid.

### 5-(2-Bromoethyl)-2,2-diethyl-1,3-dioxan

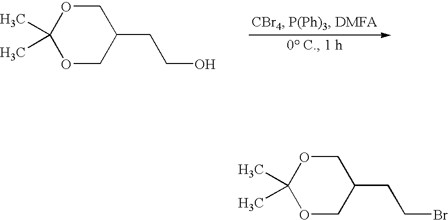

The solution of 24 g (0.15 mol) of 5-(2-hydroxyethyl)-2,2-dimethyl-1,3-dioxan and 76 g (0.23 mol) of carbon tetrabromide in 500 ml of N,N-dimethylformamide was placed in an ice bath (0-+5° C.) and rapidly stirred while 60 g (0.23 mol) of triphenylphosphine was added. The solution was stirred for one hour. The solution was then diluted with saturated aqueous sodium bicarbonate (200 ml) followed by water (300 ml), and was extracted with hexane (3×200 ml). The combined organic layers were dried over magnesium sulphate, filtered and the solvent removed under reduced pressure. The residue was placed under vacuum (0.5 mm) with a slow stream of dry argon for two hours to remove bromoform. The residue was dissolved in hexane. The solution was filtered and the solvent removed to afford 26 g (81%) of 5-(2-bromoethyl)-2,2-dimethyl-1,3-dioxan as a clear colorless liquid.

### 1-[2-(2,2-Dimethyl-1,3-dioxan-5-yl)-ethyl]-5-methyluracil

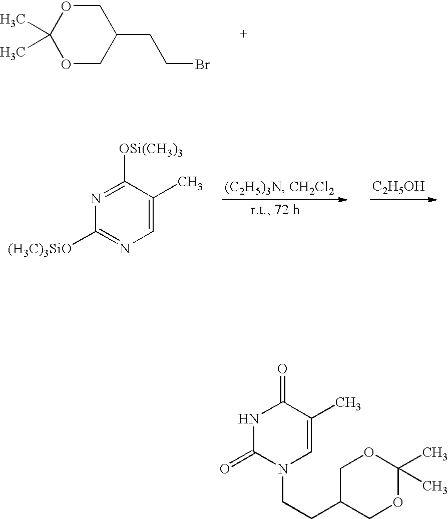

A mixture of 6.3 g (50 mmol) of thymine, 100 mL of hexamethyldisilazane, and 1 mL of trimethylsilylchloride was refluxed with stirring under nitrogen for 20 hours. The resultant solution was spin evaporated in vacuo to an oil. To the residual oil in 50 mL of dichloromethane was added 11 g (50 mmol) of 5-(2-bromoethyl)-2,2-dimethyl-1,3-dioxan. The solution was cooled on ice and 11 mL (80 mmol) of triethylamine in 50 mL of dichloromethane was added rapidly dropwise. The resultant solution was stirred at ambient temperature for 72 hours. The reaction was poured over 300 ml of aqueous (1:1) ethanol solution and shaken in a separatory funnel. The resultant mixture was diluted with 500 mL of water. The organic layer was separated, filtered and spin evaporated in vacuo.

The residue crystallized under cyclohexane containing some ethyl acetate: yield 7.3 g (54%), which contained some impurities. Several recrystallizations from ethyl acetate gave pure 1-[2-(2,2-dimethyl-1,3-dioxan-5-yl)-ethyl]-5-methyluracil: yield 6.1 g (45%). LCMS purity—98.4%

### 1-[4-Hydroxy-3-(hydroxymethyl)but-1-yl]-5-methyluracil

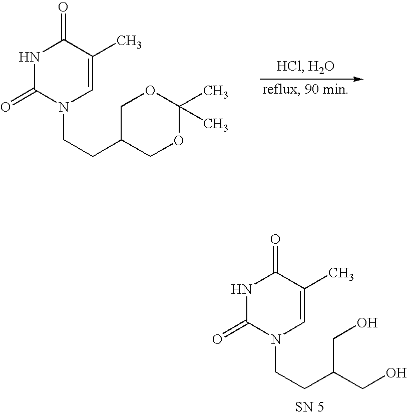

A solution of 6.1 g (23 mmol) of (1-[2-(2,2-dimethyl-1,3-dioxan-5-yl)-ethyl]-5-methyluracil in 30 mL of 2 M hydrochloric acid was heated under reflux for 90 minutes The solution was neutralized by addition of aqueous NaOH (10%) and then cooled to 10° C. The solution was filtered, and the solid was washed with water to afford 3.5 g (67%) of 1-[hydroxy-3-(hydroxymethyl)but-1-yl]-5-methyluracil. LCMS purity—97.2%.

(f) Synthesis of Formula (VI) or SN6

### 9-[2-(2,2-Dimethyl-1,3-dioxan-5-yl)-ethyl]-adenine

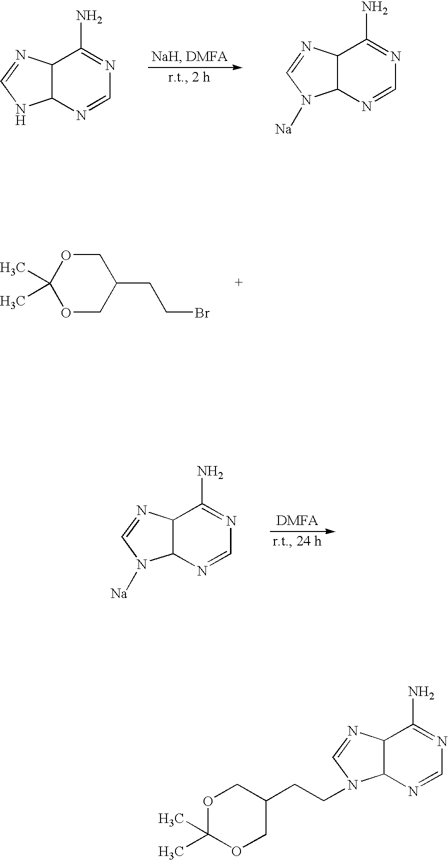

A 60% dispersion of sodium hydride in paraffin, (1.67 g, 42 mmol) was washed with hexane (3×50 mL) then suspended in 150 mL of dry DMF at 0° C. To this was added adenine (5.0 g, 37 mmol) slowly with stirring. On completion of addition the reaction mixture was warmed to room temperature and stirred for two hours before 12.4 g (56 mmol, 1.5 eq.) of 5-(2-bromoethyl)-2,2-dimethyl-1,3-dioxan was added over three hours with continual stirring. The reaction mixture was stirred at room temperature for a further 24 hours then the solution was concentrated under reduced pressure to a paste. Water (100 mL) was added and the precipitate collected and crystallized from 1-propanol, to give the 9-[2-(2,2-dimethyl-1,3-dioxan-5-yl)-ethyl]-adenine (4.8 g, 20.46%).

LCMS purity after recrystallization—98.1%.

### 9-[4-Hydroxy-3-(hydroxymethyl)but-1-yl]-adenine

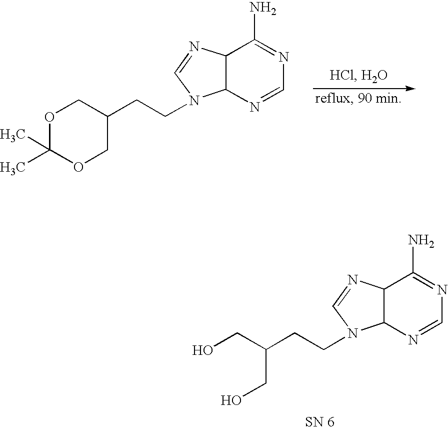

A solution of 4.3 g (15.5 mmol) of 9-[2-(2,2-dimethyl-1,3-dioxan-5-yl)-ethyl]-adenine in 20 mL of 2 M hydrochloric acid was heated under reflux for 90 minutes The solution was neutralized by addition of aqueous NaOH (10%) and then cooled to 10° C. The solution was filtered, and the solid was washed with water to afford 2.6 g (71%) of 9-[4-hydroxy-3-(hydroxymethyl)but-1-yl]-adenine. LCMS purity—97.7%.

(g) Synthesis of Valine Ester of SN1 (VSN 1)

### 2-[(5-Methyluracil-1-yl)methoxy]-ethyl N-[(benzyloxy)carbonyl]-L-valinate

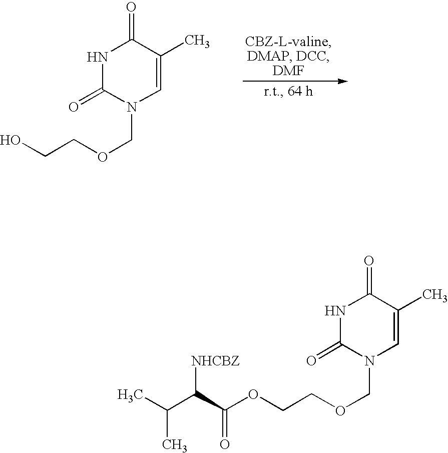

A mixture of 3 g of 1-[(2-hydroxyethoxy)methyl]-S-methyluracil and 200 ml of dry dimethylformamide was warmed to 60° C. to give a solution. 4 g of N-[(benzyloxy)carbonyl]-L-valine, 400 mg 4-dimethylaminopyridine and 4 g of dicyclohexylcarbodiimide were added to the warm solution. The resulting solution was allowed to cool to room temperature and stirred for 16 hours. The reaction mixture was recharged with the another portion of N-[(benzyloxy)carbonyl]-L-valine, 4-dimethylaminopyridine and dicyclohexylcarbodiimide and stirred at room temperature for two days. The suspension was filtered, the colorless filtrate was concentrated under reduced pressure and the residue was dissolved in methanol/dichloromethane and purified by flash chromatography on silica gel, eluting with 10% methanol/dichloromethane to yield the 2-[(5-methyluracil-1-yl)methoxy]-ethyl N-[(benzyloxy)carbonyl]-L-valinate as 5.5 g (85%) of a white solid.

### 2-[(5-Methyluracil-1-yl)methoxy]-ethyl L-valinate hydrochloride monohydrate

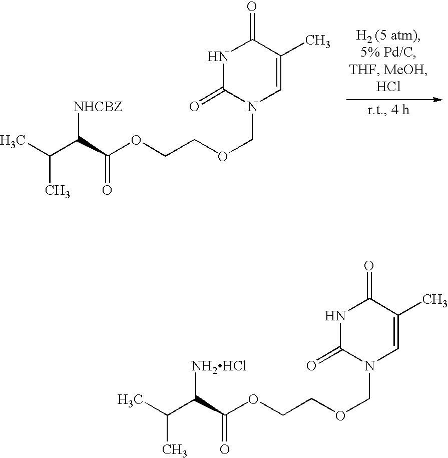

A mixture of 4 g of 2-[(5-methyluracil-1-yl)methoxy]-ethyl N-[(benzyloxy)carbonyl]-L-valinate, 500 mg 5% palladium on carbon, 100 ml of methanol, 100 ml of tetrahydrofuran and 10 ml of 0.5 M aqueous HCl solution was shaken under 5 atm H₂ for 4 hours. The reaction mixture was filtered and the filtrate was concentrated and dried to give the title compound as a white solid. A solid was crystallised from aqueous ethanol (1:3) to yield the 1.92 g (62%) 2-[(5-methyluracil-1-yl)methoxy]-ethyl L-valinate hydrochloride monohydrate as a white powder.

NMR (500 MHz, DMSO-d₆) δ, 0.96 (d, 3. H), 1.02 (d, 3H), 1.79 (s, 3H), 2.21 (m, 1H), 3.22 (m, 3H), 4.25-4.34 (m, 2H), 5.09 (s, 2H), 7.52 (s, 1H), 8.68 (br s, 3H), 11.23 (s, 1H).

(h) Synthesis of Valine Ester of SN2 (VSN 2)

### 2-[(Adenine-9-yl)methoxy]-ethyl N-[(benzyloxy)carbonyl]-L-valinate

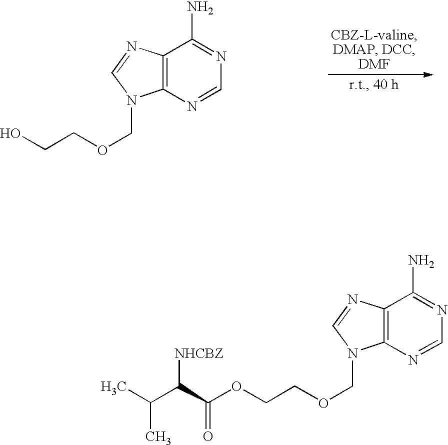

A mixture of 4 g of 9-[(2-hydroxyethoxy)methyl]-adenine and 200 ml of dry dimethylformamide was warmed to 60° C. to give a solution 5 g of N-[(benzyloxy)carbonyl]-L-valine, 500 mg 4-dimethylaminopyridine and 5 g of dicyclohexylcarbodiimide were added to the warm solution. The resulting solution was allowed to cool to room temperature and stirred for 16 hours. The reaction mixture was recharged with the another portion of N-[(benzyloxy)carbonyl]-L-valine, 4-dimethylaminopyridine and dicyclohexylcarbodiimide and stirred at room temperature for 24 hours. The suspension was filtered, the colorless filtrate was concentrated under reduced pressure and the residue was dissolved in dichloromethane and purified by flash chromatography on silica, eluting with dichloromethane to yield 2-[(adenine-9-yl)methoxy]-ethyl N-[(benzyloxy)carbonyl]-L-valinate as 6.7 g (79%) of a white solid.

### 2-[(Adenine-9-yl)methoxy]-ethyl L-Valinate Hydrochloride Monohydrate

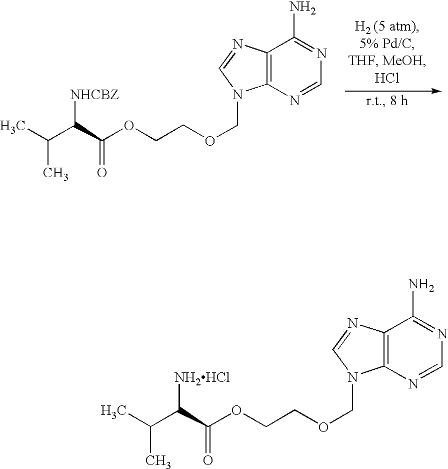

A mixture of 5 g of 2-[(adenine-9-yl)methoxy]-ethyl N-[(benzyloxy)carbonyl]-L-valinate., 700 mg 5% palladium on carbon, 130 ml of methanol, 130 ml of tetrahydrofuran and 20 ml of 0.5 M aqueous HCl solution was shaken under 5 atm H₂ for 8 hours. The reaction mixture was filtered and the filtrate was concentrated and dried to give the title compound as a white solid. A solid was crystallised from ethanol to yield the 2.24 g (58% e) 2-[(adenine-9-yl)methoxy]ethyl L-valinate hydrochloride monohydrate as a white powder.

NMR (500 MHz, DMSO-d₆) δ, 0.91 (d, 3H), 0.95 (d, 3H), 2.18 (m, 1H), 3.68 (s, 2H), 3.81 (br. s, 2H), 4.26 (m, 2H), 5.72 (s, 2H), 8.55 (s, 1H), 8.71-8.77 (m, 3H), 9.03 (br. s, 1H), 9.68 (br. s, 1H).

### Example 10

### Biological Assays Involving SN Compounds

(a) Cytotoxcity Assay: The cytotoxic effects of compounds of the invention was determined using pharmacological models that are well known to the art, i.e., MTT-microtiter plate tetrazolium cytotoxicity Assay. Specifically, cytotoxicity assays were performed using the MIT assay procedure described in Mosmann, 1983, Rapid calorimetric assay for cellular growth and survival: application to proliferation and cytotoxicity assays, J Immunol Methods, 65:55-63. Briefly, the assay was performed by using 96-well microtiter plates plated at 10⁴ MDCK (ATCC) cells/well, in 200 mL of growth medium.

For determination of IC₅₀ cells were exposed continuously for 2 days to varying concentrations of SN1, SN2, SN3, SN4 and valine ester of SN1. MIT assays were performed at the end of the 2nd day. Each assay was performed with a control that did not contain any drug. All assays were performed at least 2 times in 3 replicate wells.

The IC₅₀ cytotoxic doses were calculated as following (micrograms/ml):

SN1-750

SN2-750

SN3->1000

SN4->1000

VSN1->1000.

(b) In Vitro Assays:

Induction of telomere shortening, G2 arrest and apoptosis in telomerase positive cancer cells have been carried out as described below.

Both telomerase positive (HeLa) telomerase negative (U-2 OS) cell lines were used. Appropriate assays were performed to detect and confirm telomerase/L1RT specific activity in these cells.

The cell lines were treated with therapeutic concentrations of SN 1 (1.5 μM) or SN 2 (1.5 μM) to demonstrate that telomeric DNA synthesis could be inhibited within the cells, and thereby induce telomere shortening. Telomere length in SN 1 or SN 2 treated and untreated cell lines was measured by flow cytometry with a telomere—specific peptide nucleic acid PNA) probe. To determine cell cycle distribution, cells were stained with propidium iodide (PI). After 14 days of treatments, HeLa cells demonstrated telomere shortening, massive apoptosis and G2 arrest (FIG. 7A) but not the (U-2 OS) cells (FIG. 7B).

To demonstrate changes in cell cycle distribution, HeLa and U-2 OS cells were separately treated with SN 1 and SN 2 for 14 days stained with PI, and analyzed by flow cytometry simultaneously. Results show G2 arrest of cell cycle.

The U-2 OS (osteosarcoma) and Hela (uterine cervix) cell lines used in this study were obtained from American Type Culture Collection (Rockville, Md.). Cells were cultured in D-MEM media supplemented with 10% fetal calf serum at 37° C. in a humidified atmosphere of 5% CO₂. For treatment of the cells with SN 1 or SN 2, the media was supplemented with 1.5 μM of SN1 or SN2.

Real time TRAP assay was performed as described (Wege et al., SYBR Green real-time telomeric repeat amplification protocol for the rapid quantification of telomerase activity. Nucleic Acids Res. 2003; 31(2):E3-3).

For telomere length measurement by flow cytometry, cells were stained with telomere specific FITC conjugated (C₃TA₂)₃ PNA (Applied Biosystems) probe and contra-stained with 0.06 μg/ml PI as described by Rufer, N., Dragowska, W., Thornbury G., Roosnek, E., Lansdorp P. M. Telomere length dynamics in human lymphocyte subpopulations were measured by flow cytometry. Nat. Biotechnol. 16, 743-747 (1998)).

Thus, it has been demonstrated herein that the nucleoside analogs SN 1 and SN 2 cause decrease in telomere lengths in telomerase positive cells. However, useful inhibitory compounds are not believed to be limited in any way to the specific compounds or nucleotide analogs and derivatives specifically exemplified above. In fact, it may prove to be the case that the most useful pharmacological compounds designed and synthesized in light of this disclosure will be second generation derivatives or further-chemically-modified acyclic nucleoside analogs.

(c) In Vivo Assays:

In Vivo Assays using the compounds of the formulas (I) or (II) in combination with AZT and ddI were carried out as follows:

Mouse hepatoma MH22A was purchased from the tissue culture collection of the Institute of Cytology Russian Academy of Medical Science, Petersburg, Russia). The frozen cells were defrosted, transferred into the culture medium MEM supplemented with 10% foetal calf serum. The cells were grown at 37° C. under a humidified atmosphere of 5% CO₂. To determine the telomerase status of MH22A cells, these cells were analysed in real time TRAP assay as described above using HeLa cells as positive control. The results clearly showed that MH22A cells are telomersse positive.

Eight weeks old male C3HA inbred mice (immunocometent mice) were purchased from Laboratory Animals Breeding Facility of Russian Academy of Medical Science (Rappalovo, Leningrad region). About 2×10⁵ MH22a cells were injected s.c. into back flank of 80 mice. Two weeks after the injections, 66 mice showing actively growing tumors (2 mm to 1 cm diameter) were selected and randomly divided into different experimental groups as set forth below:

Control group—18 mice

SN-1 (0.5 mg/ml)+AZT (0.05 mg/ml)+ddI (0.033 mg/ml)−10 mice

SN-2 (0.5 mg/ml)+AZT (0.05 mg/ml)+ddI (0.033 mg/ml)−10 mice

Valtrex® (0.083 mg/ml)+AZT (0.05 mg/ml)+ddI (0.033 mg/ml)−20 mice

Valtrex® (manufactured by GlaxoSmithKline) is a prodrug of acyclovir, which is an acyclic nucleoside analog. Didanosine (ddI) and AZT are non-acyclic nucleoside analogs. The drugs, at the concentrations indicated above, were administered to the mice in drinking water. The consumption of drinking water or solutions of the drugs was about 4 ml per mouse per day. In all groups, mice died from progressive tumors (2.0 cm-2.5 cm in diameter) with the exception of mice in Valtrex®+AZT+DDI group. In this group of 20 mice, only 5 developed tumors comparable in size with the control group, 7 had no tumors, and 8 had small tumors up to 1 cm only. This group also had some mice dying during the course of the treatment period. Part of the mortality rate may be attributed to immune reactions, since the mice used in the experiments were immunocompetent. The following results were noted in 5 weeks following the treatments with different combination of nucleoside analogs.

Control Group: The control received no nucleoside analog. As a result, all mice died, at the time of death, tumors were 3.0 cm-35 cm in diameter.

SN-1+AZT+ddI: A total of 5 mice were alive of which 3 were without tumors. The tumors in the remaining two mice were 0.7 cm and 3.5 cm, respectively.

SN-2+AZT+ddI: A total of 7 mice were alive of which 2 were without tumors. The tumors in four mice were 3.0 cm to 3.5 cm in diameter and that in the remaining one mouse was 1.0 cm diameter.

Valtrex®+AZT+ddI: A total of 5 mice were alive of which 3 were without tumors. The tumors in the remaining two mice were 1.0 cm and 0.5 cm, respectively. In this group, a total of 15 mice died, of which 5 died from tumor growth, the remaining 10 mice died with small tumors or had no tumors.

In Vivo assays using the compounds of the formulas (I) to (IV) and valine esters of formulas I and II each in a triple cocktail with other nucleoside analogs and tumor in combination with AZT and ddI were carried out as follows:

8-10 weeks old male C3HA inbred mice (immunocometent mice) were purchased from Laboratory Animals Breeding Facility of Russian Academy of Medical Science (Rappalovo, Leningrad region). 3×10⁵ MH22a cells were injected s.c. into back flank of 200 mice. Ten days after the injections, mice bearing developing tumors (14.15 mm³) (the tumor volume calculated using the formula π/6 DmaxDmin²) were selected and randomly divided into different experimental groups as set forth below:

Cocktail 1—Valacyclovir (0.166 mg/ml)+AZT (0.1 mg/ml)+ddI (0.066 mg/ml)−40 mice;

Cocktail 2—SN-1 (1 mg/ml)+AZT (0.1 mg/ml)+ddI (0.066 mg/ml)−10 mice;

Cocktail 3—SN-3 (1 mg/ml)+AZT (0.1 mg/ml)+ddI (0.066 mg/ml)−10 mice;

Cocktail 4—SN4 (1 mg/ml)+AZT (0.1 mg/ml)+ddI (0.066 mg/ml)−10 mice;

cocktail 5—Val-SN-1 (0.166 mg/ml)+AZT (0.1 mg/ml)+ddI (0.066 mg/ml)−20 mice; and

Cocktail 6—Val-SN-2 (0.166 mg/ml)+AZT (0.1 mg/ml)+ddI (0.066 mg/ml)−20 mice; and

Control group—Capecitabine (Xeloda®) (1 mg/ml) in drinking water-mice.

Valacyclovir (Valtrex® manufactured by GlaxoSmithKline) is a prodrug of acyclovir, which is an acyclic nucleoside analog. Didanosine (ddI) and AZT are non-acyclic-nucleoside analogs. The drugs, at the concentrations indicated above, were administered to the mice in drinking water. Two weeks following the cocktail treatments Xeloda® (1 mg/ml) was added to each of the cocktails and continued for the remaining experimental period. In the control group, mice were treated only with Xeloda® (1 mg/ml) in drinking water and this treatment started at the same time as the Xeloda® treatments in the cocktail groups. The consumption of drinking water or solutions of the drugs was on an average 2.5 ml per mouse per day. Tumor volumes (mm³) in each injected mice were calculated, using the formula π/6 DmaxDmin², every week for four consecutive weeks following the commencement of the treatment with the drugs. In all of the experimental groups, except the group with SN4 combination, mice died from progressive tumors

The following results were noted in 4 weeks following the treatments with cocktails 16 and Xeloda® as described above.

EXAMPLE 10 TABLE 1
Efficacy of in vivo treatments.

| | | | Number | Number died |
|---|---|---|---|---|
| | | | showing | during the 4 |
| | Number of | Number | tumors after | week |
| Drug(s) in | mice | tumor- | four weeks of | treatment |
| drinking water | treated | free | treatment | period |
| Cocktail 1 | 40 | 8 | 22 | 10 |
| Cocktail 2 | 10 | 1 | 8 | 1 |
| Cocktail 3 | 10 | 1 | 4 | 5 |
| Cocktail 4 | 10 | 3 | 7 | 0 |
| Cocktail 5 | 20 | 4 | 9 | 7 |
| Cocktail 6 | 20 | 3 | 10 | 7 |
| Xetoda ® | 8 | 0 | 6 | 2 |

EXAMPLE 10 TABLE 2
Tumor volume patterns in mice at the end of the experimental. period after
treatments with various drug(s) indicated in Example 10, Table 1.

| | Cocktail | Cocktail | Cocktail | Cocktail | Cocktail | Cocktail | |
|---|---|---|---|---|---|---|---|
| Mouse | 1 | 2 | 3 | 4 | 5 | 6 | Xeloda ® |
| 1 | 0.524 | 33.54 | 1056 | 1151 | 0.131 | 0.524 | 1152 |
| 2 | 0.524 | 1541 | 1769 | 1438 | 1768 | 91.7 | 2146 |
| 3 | 65.5 | 4244 | 2358 | 3565 | 2004 | 1151 | 4192 |
| 4 | 524 | 5030 | 5856 | 3786 | 2142 | 3396 | 6288 |
| 5 | 905.5 | 5240 | | 4240 | 2142 | 4821 | 6288 |
| 6 | 2358 | 7126 | | 8451 | 2410 | 4853 | 10480 |
| 7 | 2415 | 7336 | | 9210 | 3079 | 4853 | |
| 8 | 3396 | 8369 | | | 3904 | 7101 | |
| 9 | 3594 | | | | 6596 | 8316 | |
| 10 | 3594 | | | | | 15091 | |
| 11 | 4075 | | | | | | |
| 12 | 4192 | | | | | | |
| 13 | 4192 | | | | | | |
| 14 | 4351 | | | | | | |
| 15 | 4540 | | | | | | |
| 16 | 5240 | | | | | | |
| 17 | 5450 | | | | | | |
| 18 | 5580 | | | | | | |
| 19 | 9564 | | | | | | |
| 20 | 9825 | | | | | | |
| 21 | 10314 | | | | | | |
| 22 | 14148 | | | | | | |

Only the measurements of tumor-bearing mice from Example 10, Table 1, are included in this table.

All publications, patents and patent applications mentioned in the specification are indicative of the level of those skilled in the art to which this invention pertains. All publications, patents and patent applications are herein incorporated by reference to the same extent as if each individual publication or patent application was specifically and individually indicated to be incorporated by reference.

The foregoing specification teaches the principles of the present invention, with description of the preferred embodiments, and with examples provided for the purpose of illustration, so as to enable any person skilled in the art to make and use the present invention. The various modifications to these embodiments will be readily apparent to those skilled in the art, and the generic principles defined herein may be applied to other embodiments without the use of the inventive faculty. Thus, the present invention is not intended to be limited to the embodiments shown herein but is to be accorded the widest scope consistent with the principles and novel features disclosed herein and the following claims and its equivalents.

1. A method of treatment comprising administering a background therapy.

2. The method of claim 1, wherein the treatment is a treatment of cancer in a subject.

3. The method of claim 2, wherein the subject is a human.

4. The method of claim 3, wherein the background therapy comprises administering therapeutically effective amount of a triple cocktail in a pharmaceutically acceptable carrier.

5. The method of claim 4, wherein the triple cocktail comprises an acyclic nucleoside analog.

6-12. (canceled)

13. The method of claim 1 further comprising administering background therapy in combination with another anti-proliferative therapy.

14. The method of claim 13, wherein the another anti-proliferative therapy is a DNA damaging therapy.

15-20. (canceled)

21. A method of sensitizing a mammal to another anti-cancer therapy or another anti-proliferative therapy comprising administering a sensitizing effective amount of a double cocktail or a triple cocktail in a pharmaceutically acceptable carrier.

22-30. (canceled)

31. A method of ameliorating a side effect of another anti-cancer therapy or another anti-proliferative therapy in a mammal comprising administering a sensitizing effective amount of a double cocktail or a triple cocktail, in a pharmaceutically acceptable carrier, through a period during which said another therapy is withheld for a time sufficient for the mammal to recover from or correct the side effect.

32-33. (canceled)

34. A method of treating cancer comprising administering a background therapy to a patient, wherein the therapy comprises administering therapeutically effective amount of a triple cocktail in combination with another anti-proliferative therapy.

35. A method for treating a subject having a condition characterized by an abnormal mammalian cell proliferation, comprising: administering to the subject in need of such treatment in an amount effective to inhibit the proliferation, a double cocktail or a triple cocktail, and wherein the condition is further characterized by the abnormally proliferating cells exhibiting telomere maintenance in successive cell divisions as compared to normal cells.

36. A method of inhibiting one or more reverse transcriptases in cells of a mammal in need of such treatment comprising administering to the mammal an effective amount of a double cocktail or a triple cocktail in a pharmaceutically acceptable carrier.

37. A method of inducing tumor cell apoptosis in a mammal in need thereof, comprising: administering to the mammal a therapeutically effective amount of a double cocktail or a triple cocktail in a pharmaceutically acceptable carrier.

38-39. (canceled)

40. A method for inducing apoptosis in a cancer cell comprising, contacting the tumor cell with a triple cocktail in a pharmaceutically acceptable carrier such that induction of apoptosis occurs.

41-42. (canceled)

43. A combination of compounds comprising an effective amount of
a first nucleoside analog or a prodrug thereof that is a telomerase reverse transcriptase (TERT) inhibitor,
a second nucleoside analog or a prodrug thereof that is a Line-1 retrotransposon encoded reverse transcriptase (L1RT) inhibitor, and optionally
a third nucleoside analog that is an inhibitor of a reverse transcriptase (RT), the RT being a non-TERT and non-L1RT, wherein the second nucleoside analog or prodrug thereof is not the same as the first nucleoside analog or prodrug thereof, wherein the third nucleoside analog or prodrug thereof is not the same as the first and second nucleoside analogs or prodrugs thereof.

44. The combination of claim 43, wherein the first, second and third nucleoside analogs or the corresponding prodrugs are in the form of three separate pharmaceutical compositions or in the form of a single pharmaceutical composition.

45. The combination of claim 44, wherein the first nucleoside analog is an acyclic nucleoside analog or a prodrug thereof, wherein the second nucleoside analog is an acyclic nucleoside analog or a prodrug thereof or azido-2′,3′-dideoxythymidine (AZT) or a prodrug of AZT, wherein the third nucleoside analog is 2′,3′-dideoxyinosine (ddI).

46. A method of treating a human patient suffering from cancer comprising administering to the human patient the combination of claim 45.

47-53. (canceled)

54. A medicinal cocktail comprising, in a pharmaceutically acceptable carrier, a combined therapeutically effective amount of
an acyclic nucleoside analog or prodrug thereof;
azido-2′,3′-dideoxythymidine (AZT); and
2′,3′-dideoxyinosine (ddI).

55. A thymine or an adenine derivative of the formula selected from the group consisting of formulas (I), (II), (III), (IV), (V) and (VI) or a physiologically acceptable salt, an optical isomer or a pro-drug thereof.

56. The derivative according to claim 55 is 1-(2-hydroxyethoxymethyl) thymine.

57. The derivative according to claim 55 is 9-(2-hydroxyethoxymethyl) adenine.

58-60. (canceled)

61. A pharmaceutical preparation comprising as an active ingredient a compound of the formula (I), (II), (III), (IV), (V) or (VI), or a physiologically acceptable salt or an optical isomer thereof; in conjunction with a pharmaceutically acceptable carrier.

62-63. (canceled)

64. A method for the treatment of cancer in an animal or human host in need of treatment, comprising administering a therapeutically effective amount of a composition comprising as an active ingredient a compound of the formula (I), (II), (III), (IV), (V) or (VI), or a physiologically acceptable salt or an optical isomer thereof, in conjunction with AZT and didanosine in a pharmaceutically acceptable carrier.

65. The method according to claim 64 comprising administering a therapeutically effective amount of the formula I or a physiologically acceptable salt thereof.

66. The method according to claim 64 comprising administering a therapeutically effective amount of the formula II or a physiologically acceptable salt thereof.

67-71. (canceled)

72. A method of inducing tumor cell apoptosis in a mammal in need thereof, comprising:
administering to the mammal a therapeutically effective amount of a a compound of the formula (I), (II), (III), (IV), (V) or (VI), or a physiologically acceptable salt or an optical isomer thereof, optionally in conjunction with AZT and didanosine in a pharmaceutically acceptable carrier; and
administering another anticancer nucleoside analog and, optionally, an anticancer agent therewith.

73. The method of claim 72, wherein the compound of the formula (I), (II), (III), (IV), (V) or (VI), and the other anticancer nucleoside analog and anticancer agent are administered as a cocktail.

74. The method of claim 72 wherein the compound of the formula (I), (II), (III), (IV), (V) or (VI), and the other anticancer nucleoside analog and anticancer agent are administered in separate unit dosage forms.

75. (canceled)
